(19) United States
(12) Patent Application Publication
(10) Pub. No.: US 2026/0018282 A1
(43) Pub. Date: Jan. 15, 2026

(54) CAREGIVER ASSISTANCE SYSTEM

(71) Applicant: Stryker Corporation, Portage, MI (US)

(72) Inventors: Thomas Joseph Durlach, Portage, MI (US); Vinod Prakash Bhatt, Gurugram, Haryana (IN); Akshay Kumar Sharma, West Sagarpur, New Delhi (IN)

(21) Appl. No.: 19/123,448

(22) PCT Filed: Oct. 31, 2023
(86) PCT No.: PCT/US2023/036362

§ 371 Date: Apr. 23, 2025

(30) Foreign Application Priority Data
Oct. 31, 2022 (IN) 202211062036

(51) Int. Cl. G16H 40/67; A61G 1/04; A61G 7/05; G16H 40/20

(52) U.S. Cl. CPC G16H 40/67; A61G 1/04; A61G 7/05; G16H 40/20; A61G 2203/20; A61G 2203/44

(57) ABSTRACT

A software application executed by one or more computing devices (e.g. a server, mobile device, and/or stationary display device) instructs the computing device to receive status conditions regarding a current state of a plurality of components of a patient support apparatus; to receive priority assignments for a plurality of undesired conditions from a user interface; to use the status conditions to determine a set of undesired conditions that currently exist for the patient support apparatus; and if the set contains multiple undesired conditions, to display a specific indicator that specifically identifies only the highest undesired condition. The software application may also instruct the display device to display a generic indicator for lower priority conditions, to display a weight-recorded icon, and/or to display a missing-weight icon. The software application may also accept undesired condition definitions based on patient support apparatus type or healthcare facility departments.

### BACKGROUND

The present disclosure relates to patient support apparatuses, such as beds, cots, stretchers, recliners, or the like. More specifically, the present disclosure relates to a system for sharing information regarding patient support apparatuses with caregivers who are positioned remotely from the patient support apparatuses.

Hospitals typically expect nurses and/or other caregivers to perform a variety of different duties when caring for patients. These duties include administering medications and/or therapies, taking vital sign readings, installing and removing IV drips, taking blood samples, ensuring patient compliance with prescribed activities and/or medications, assisting the patient into and out of bed, regularly visiting the patient, documenting one or more of these activities, and generally being responsive to the patient's needs. In addition to these duties, caregivers are also generally expected to configure the patient's bed to be in a defined state desired by the healthcare facility for patient safety and/or for other purposes. This extra duty of overseeing the state of the bed adds to the caregiver's workload.

### SUMMARY

According to various embodiments, a tool is provided for assisting caregivers with their task of ensuring that the patient support apparatuses of their patients are in their desired states. The tool may take on the form of a software application executed by a server in communication with the patient support apparatuses, or it may take on other forms. According to some embodiments, the tool provides an easy-to-understand dashboard of the status of the patient support apparatuses in a manner that prioritizes the information of greatest interest to the healthcare facility while helping to reduce alarm fatigue for the caregivers. In some embodiments, the tool also helps ensure that caregiver comply with the healthcare facility's protocols regarding the weighing of patients. In some embodiments, the tool is easily customizable for not only individual patient support apparatuses, but also groups of patient support apparatuses, such as those positioned within a particular location, wing, department, etc. of a healthcare facility, and/or particular types or models of patient support apparatuses. Still other features and functions of the tool are included, as discussed in greater detail below.

According to a first embodiment of the present disclosure, a software application is provided that is adapted to, when executed by a processor of a computing device, cause the computing device to perform the following: receive status conditions from a patient support apparatus that includes a current state of a plurality of components of the patient support apparatus; receive priority assignments for a plurality of undesired conditions from a user interface; use the status conditions to determine a set of undesired conditions that currently exist for the patient support apparatus; and, if the set contains multiple undesired conditions that currently exist for the patient support apparatus, perform the following: (a) select the undesired condition having the highest priority assignment from the set of undesired conditions; (b) instruct a display device to display a specific indicator that specifically identifies the selected undesired condition; and (c) instruct the display device to display a generic indicator that does not specifically identify the undesired conditions in the set that do not have the highest priority assignment. If the set contains only a single undesired condition that currently exists for the patient support apparatus, the software application is configured to cause the computing device to instruct the display device to display a single specific indicator that specifically identifies the single undesired condition.

According to another aspect of the present disclosure, a software application is provided that is embodied in a non-transitory computer readable medium and adapted, when executed by a processor of a computing device, to cause the computing device to perform the following: receive status conditions from a patient support apparatus that includes a current state of a plurality of components of the patient support apparatus; receive weight data indicating when a weight of a patient assigned to the patient support apparatus was last measured using the patient support apparatus; and instruct a display device to display a specific indicator based on the status conditions and a weight icon based on the weight data.

According to another aspect of the present disclosure, a software application is provided that is embodied in a non-transitory computer readable medium and adapted, when executed by a processor of a computing device, to cause the computing device to perform the following: receive status conditions from a patient support apparatus that includes a current state of a plurality of components of the patient support apparatus; receive condition definitions from a user interface that are based on at least one of the following: a current location of the patient support apparatus, a department to which the patient support apparatus is assigned, or a patient support apparatus type; use the undesired condition definitions and the status conditions to determine if an undesired condition currently exists for the patient support apparatus; and, if an undesired condition currently exists for the patient support apparatus, instruct a display device to display information indicative of the undesired condition, and if an undesired condition does not currently exist for the patient support apparatus, not instruct the display device to display information indicative of the undesired condition.

According to other aspects of the present disclosure, the generic indicator may be a number having a value equal to one less than a total number of undesired conditions in the set of undesired conditions.

In some aspects, the specific indicator includes a graphical symbol.

The plurality of undesired conditions, in some aspects, includes at least one of the following: a brake on the patient support apparatus not set, a position of one or more siderails on the patient support apparatus not being in a desired position, an exit detection system of the patient support apparatus not being armed, or a height of a litter frame of the patient support apparatus not being at a desired height.

The software application, in some aspects, is further adapted to instruct the computer device to receive location data from the patient support apparatus, to use the location data to determine a room number in which the patient support apparatus is currently located, and to instruct the display device to display the room number on the display device.

In some aspects, the software application is further configured to instruct the computer device to perform the following: receive additional status conditions from a plurality of additional patient support apparatuses, the additional status conditions including a current state of a plurality of components of each of the plurality of additional patient support apparatuses; use the additional status conditions to determine an additional set of undesired conditions that currently exist for each one of the plurality of additional patient support apparatuses; and for each additional patient support apparatus in the plurality of additional patient support apparatuses, if the additional set contains multiple undesired conditions that currently exist for the additional patient support apparatus, perform the following: (a) select the undesired condition having the highest assignment from the additional set of undesired conditions; (b) instruct the display device to display a specific indicator that specifically identifies the selected undesired condition; and (c) instruct the display device to display a generic indicator that does not specifically identify the undesired conditions in the additional set that do not have the highest assignment. The software application is also configured to cause the computing device to instruct the display device to display a single specific indicator that specifically identifies the single undesired condition if the additional set contains only a single undesired condition that currently exists for the additional patient support apparatus.

In some aspects, the computing device is a server communicatively coupled to the display device by a computer network.

The server, in some aspects, is communicatively coupled to the display device by a WiFi connection.

The display device, in some aspects, is a smart phone, a tablet computer, a television, or a laptop computer.

In some aspects, the software application is further adapted to instruct the display device to display an enclosed area on a display of the display device, wherein the enclosed area corresponds to a particular room of a healthcare facility and the software application instructs the display device to display the specific indicator within the enclosed area.

The software application, in some aspects, is further adapted to instruct the display device to display the generic indicator within the enclosed area.

In some aspects, the software application is further adapted to instruct the computing device to send an alert to a caregiver badge, wherein the alert notifies a caregiver associated with the caregiver badge of the selected undesired condition but does not notify the caregiver of the undesired conditions in the set that do not have the highest priority assignment.

The software application, in some aspects, is further adapted to instruct the computing device to receive undesired condition definitions from the user interface, wherein the undesired condition definitions define what undesired conditions are applicable to the patient support apparatus.

In some aspects, the software application is further adapted to instruct the computing device to receive undesired condition definitions that are based on at least one of the following: a current location of the patient support apparatus, a department to which the patient support apparatus is assigned, a health condition of a patient, or a patient support apparatus type.

The software application, in some aspects, is further adapted to instruct the computing device to receive undesired condition definitions that are based on both a department to which the patient support apparatus is assigned and a patient support apparatus type.

The patient support apparatus type, in some aspects, defines whether the patient support apparatus is a bed or a stretcher.

In some aspects, the patient support apparatus type defines a specific model of a bed or a specific model of a stretcher.

The software application, in some embodiments, is further adapted to instruct the computing device to perform the following: (1) receive weight data indicating when a weight of a patient assigned to the patient support apparatus was last measured using the patient support apparatus, and (2) instruct the display device to display a weight icon based on the weight data.

In some aspects, the software application is further adapted to instruct the computing device to perform the following: (1) receive patient assignment data indicating when a new patient is assigned to the patient support apparatus; (2) receive weight data indicating when a weight of a patient assigned to the patient support apparatus was last measured using the patient support apparatus; (3) if the computing device does not receive the weight data within a time period after receiving the patient assignment data, instruct the display device to display a missing-weight icon; and (4) if the computing device does receive the weight data with the time period after receiving the patient assignment data, instruct the display device to display a weight-recorded icon.

In some aspects, the software application is further adapted to instruct the computing device to perform the following: (1) receive weight data indicating when a weight of a patient assigned to the patient support apparatus was last measured using the patient support apparatus; (2) determine if updated weight data has been received within a time window of receiving the weight data; (3) if the computing device has not received the updated weight data within the time window, instruct the display device to display a missing-weight icon; and (4) if the computing device has received the updated weight data with the time window, instruct the display device to display a weight-recorded icon.

The software application, in some aspects, is further adapted to instruct the computing device to receive a value defining the time period from the user interface.

In some aspects, the time window and the time period are equal.

In some aspects, the time window and the time period are not equal.

In some aspects, the software application is further adapted to instruct the computing device to receive a value defining the time period from a user interface of the computing device.

The software application, in some aspects, is further adapted to instruct the computing device to receive the weight data from the patient support apparatus.

The software application, in some aspects, is further adapted to instruct the computing device to receive the weight data from an electronic medical records server.

In some aspects, the software application is further adapted to instruct the computing device to receive the weight data from both an electronic medical records server and the patient support apparatus.

In some aspects, the software application is further adapted to instruct the computing device to receive undesired condition definitions that are based on both the department to which the patient support apparatus is assigned and the patient support apparatus type.

According to other aspects of the present disclosure, a system is provided that includes a server and a display device. The server is adapted to execute any one or more of the above described aspects of the software application, and the display device is a smart phone, a tablet computer, a television, or a laptop computer.

The software application, in some aspects, is adapted to communicate with the display device via web-browser installed on the display device.

In some aspects, the software application is adapted to communicate with the display device via a native software application installed on the display device.

Before the various embodiments disclosed herein are explained in detail, it is to be understood that the claims are not to be limited to the details of operation or to the details of construction and the arrangement of the components set forth in the following description or illustrated in the drawings. The embodiments described herein are capable of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the claims to any specific order or number of components. Nor should the use of enumeration be construed as excluding from the scope of the claims any additional steps or components that might be combined with or into the enumerated steps or components.

### BRIEF DESCRIPTION OF THE DRAWINGS

FIG. 1 is a perspective view of a patient support apparatus usable with a caregiver assistance system according to one embodiment of the disclosure;

FIG. 2 is a block diagram of a detailed set of components of the patient support apparatus of FIG. 1, as well as several devices in communication with the patient support apparatus, such as a patient support apparatus server, a display device, and a portion of a local area network;

FIG. 3 is a block diagram showing additional details of several servers that may communicate with the patient support apparatus server of FIG. 2;

FIG. 4 is an example of a dashboard screen that may be displayed by one or more display devices in communication with the patient support apparatus server of FIG. 2;

FIG. 5 is a prioritization screen that may be displayed on a display device having appropriate access to the patient support apparatus server of FIG. 2;

FIG. 6 is a rule-summary screen that may be displayed on a display device having appropriate access to the patient support apparatus server of FIG. 2;

FIG. 7 is a rule-setting screen that may be displayed on a display device having appropriate access to the patient support apparatus server of FIG. 2;

FIG. 8 is an example of a missing-weight icon that may be displayed on a dashboard screen, such as the dashboard screen of FIG. 4;

FIG. 9 is an example of a weight-recorded icon that may be displayed on a dashboard screen, such as the dashboard screen of FIG. 4; and

FIG. 10 is an example of an abbreviated dashboard screen that may be displayed on a display device, such as a smart phone or tablet computer.

### DETAILED DESCRIPTION OF THE EMBODIMENTS

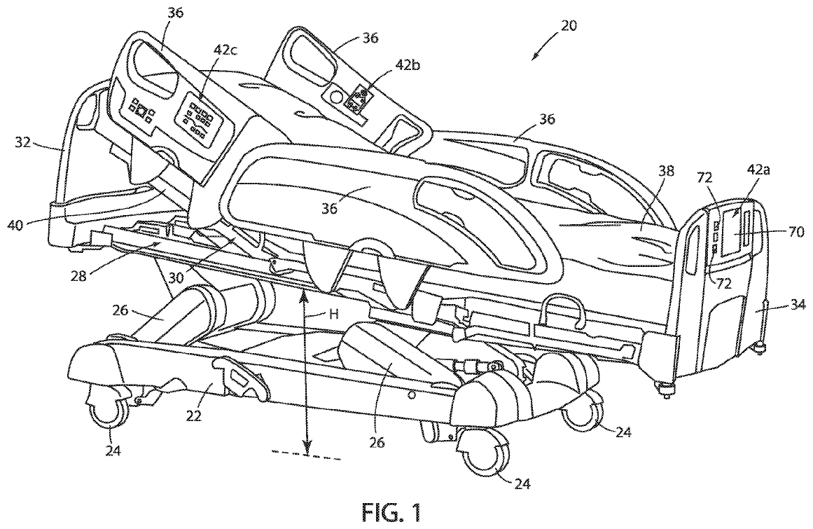

An illustrative patient support apparatus 20 usable in a caregiver assistance system according to the present disclosure is shown in FIG. 1. Although the particular form of patient support apparatus 20 illustrated in FIG. 1 is a bed adapted for use in a hospital or other medical setting, it will be understood that patient support apparatus 20 could, in different embodiments, be a cot, a stretcher, a recliner, or any other structure capable of supporting a patient while the patient is in a healthcare facility, such as, but not limited to, a hospital. For purposes of the following written description, patient support apparatus 20 will be primarily described as a bed with the understanding that the following written description applies to these other types of patient support apparatuses.

In general, patient support apparatus 20 includes a base 22 having a plurality of wheels 24, a lift subsystem comprising a pair of lifts 26 supported on the base, a litter frame 28 supported on the lifts 26, and a support deck 30 supported on the litter frame 28. Patient support apparatus 20 further includes a headboard 32, a footboard 34, and a plurality of siderails 36. Siderails 36 are all shown in a raised position in FIG. 1 but are each individually movable to a lower position in which ingress into, and egress out of, patient support apparatus 20 is not obstructed by the lowered siderails 36. In some embodiments, siderails 36 may be moved to one or more intermediate positions as well.

Lifts 26 are configured to raise and lower litter frame 28 with respect to base 22. Lifts 26 may be hydraulic actuators, electric actuators, or any other suitable device for raising and lowering litter frame 28 with respect to base 22. In the illustrated embodiment, lifts 26 are operable independently so that the tilting of litter frame 28 with respect to base 22 can also be adjusted. That is, litter frame 28 includes a head end and a foot end, each of whose height can be independently adjusted by the nearest lift 26. Patient support apparatus 20 is designed so that when an occupant lies thereon, his or her head will be positioned adjacent the head end and his or her feet will be positioned adjacent the foot end.

Litter frame 28 provides a structure for supporting support deck 30, the headboard 32, footboard 34, and siderails 36. Support deck 30 provides a support surface for a mattress 38, or other soft cushion, so that a person may lie and/or sit thereon. Support deck 30 is made of a plurality of sections, some of which are pivotable about generally horizontal pivot axes. In the embodiment shown in FIG. 1, support deck 30 includes a head section 40, which is also sometimes referred to as a Fowler section or a backrest section. Head section 40 is pivotable about a generally horizontal pivot axis between a generally horizontal orientation (not shown in FIG. 1) and a plurality of raised positions (one of which is shown in FIG. 1). Support deck 30 may include additional sections that are pivotable about one or more horizontal pivot axes, such as an upper leg or thigh section and/or a lower leg or foot section (not labeled).

Patient support apparatus 20 further includes a plurality of control panels 42 that enable a user of patient support apparatus 20, such as a patient and/or an associated caregiver, to control one or more aspects of patient support apparatus 20. In the embodiment shown in FIG. 1, patient support apparatus 20 includes a footboard control panel 42a, a pair of inner siderail control panels 42b (only one of which is visible), and a pair of outer siderail control panels 42c (only one of which is visible). Footboard control panel 42a and outer siderail control panels 42c are intended to be used by caregivers, or other authorized personnel, while inner siderail control panels 42b are intended to be used by the patient associated with patient support apparatus 20. Not all of the control panels 42 include the same controls and/or functionality. In the illustrated embodiment, footboard control panel 42a includes a substantially complete set of controls for controlling patient support apparatus 20 while control panels 42b and 42c include a selected subset of those controls. One or more of any of control panels 42a, b, and/or c may include a height adjustment control that, when activated, changes a height of litter frame 28 relative to base 22.

Control panels 42a and/or 42c may include controls for allowing a user to do one or more of the following: activate and deactivate a brake for wheels 24, arm an exit detection system 46, take a weight reading of the patient, activate and deactivate a propulsion system, and communicate with a healthcare facility computer network installed in the healthcare facility in which patient support apparatus 20 is positioned. Inner siderail control panels 42b may also include a nurse call control that enables a patient to call a nurse. A speaker and microphone are included on, or adjacent to, inner siderail control panel 42b in order to allow the patient to aurally communicate with the remotely positioned nurse.

Footboard control panel 42a is implemented in the embodiment shown in FIG. 1 as a touchscreen display 70 having a plurality of controls 72 positioned alongside the touchscreen display 70. Controls 72 may be implemented as buttons, dials, switches, or other devices. Either or both of control panels 42b or 42c may also include a display for displaying information regarding patient support apparatus 20, and such a display may be a touchscreen in some embodiments. Alternatively, any one or more of control panels 42a-c may omit a touchscreen display and instead include only dedicated controls 72, or some other form of non-display controls.

The mechanical construction of those aspects of patient support apparatus 20 not explicitly described herein may be the same as, or nearly the same as, the mechanical construction of the Model FL27 InTouch Critical Care bed manufactured and sold by Stryker Corporation of Kalamazoo, Michigan. This mechanical construction is described in greater detail in the Stryker Maintenance Manual for the Model FL27 InTouch Critical Care Bed (Version 2.4; 2131-409-002 REV B), published by Stryker Corporation of Kalamazoo, Michigan, the complete disclosure of which is incorporated herein by reference. It will be understood by those skilled in the art that those aspects of patient support apparatus 20 not explicitly described herein can alternatively be designed with other types of mechanical constructions, such as, but not limited to, those described in commonly assigned, U.S. Pat. No. 7,690,059 issued to Lemire et al., and entitled HOSPITAL BED; and/or commonly assigned U.S. Pat. publication No. 2007/0163045 filed by Becker et al. and entitled PATIENT HANDLING DEVICE INCLUDING LOCAL STATUS INDICATION, ONE-TOUCH FOWLER ANGLE ADJUSTMENT, AND POWER-ON ALARM CONFIGURATION, the complete disclosures of both of which are also hereby incorporated herein by reference.

The mechanical construction of those aspects of patient support apparatus 20 not explicitly described herein may alternatively be the same as, or nearly the same as, the mechanical construction of any of the ProCuity™ Bed Series beds manufactured and sold by Stryker Corporation of Kalamazoo, Michigan. The mechanical construction of these beds is described in greater detail in the Stryker Maintenance Manual for the ProCuity™ Bed Series beds (3009-109-002 Rev. AA.0), published by Stryker Corporation of Kalamazoo, Michigan, in April of 2021, the complete disclosure of which is incorporated herein by reference. The mechanical construction of those aspects of patient support apparatus 20 not explicitly described herein may also take on still other forms different from what is disclosed in the aforementioned references.

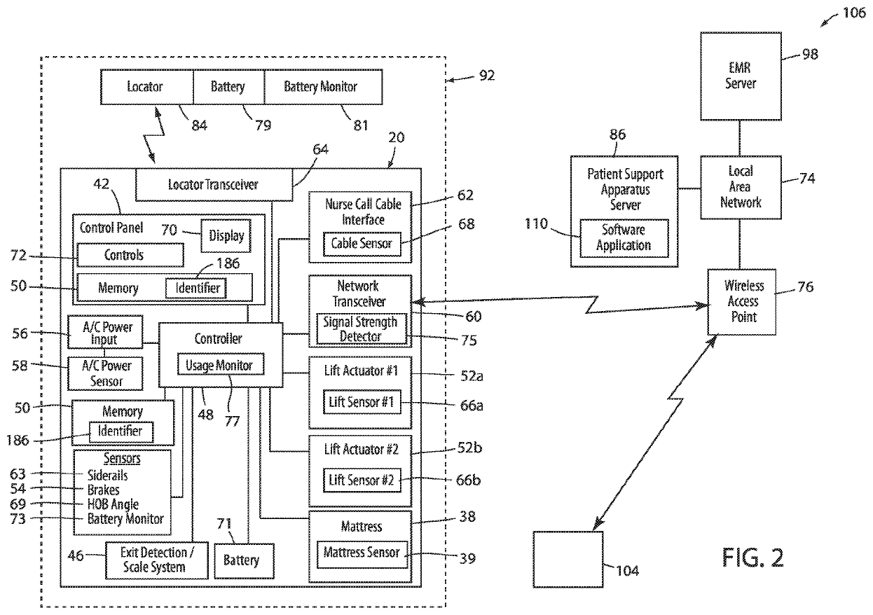

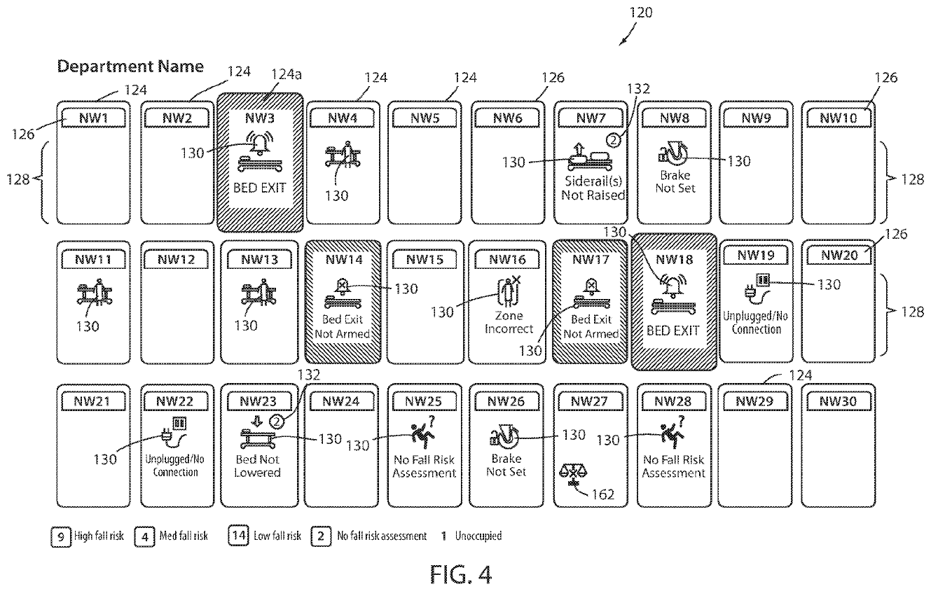

FIG. 2 illustrates a first embodiment of a caregiver assistance system 106 according to the present disclosure. Caregiver assistance system 106 includes patient support apparatus 20 in communication with a patient support apparatus server 86, and one or more display devices 104 that are adapted to communicate with patient support apparatus server 86. The patient support apparatus server 86, like all of the servers discussed herein, includes one or more conventional microprocessors. Patient support apparatus server 86 is adapted to execute a software application 110 that receives various data from one or more patient support apparatuses 20 and forwards some, or all, of this data to one or more display devices 104 for display thereon. As will be discussed in greater detail below with respect to FIG. 4, software application 110 may communicate with a plurality of other servers on a local area network 74 of the healthcare facility and use those communications to obtain some of the information it needs to perform some of the caregiver assistance functions described herein.

FIG. 2 illustrates in greater detail some of the internal components of patient support apparatus 20. As shown therein, patient support apparatus 20 includes a controller 48, a memory 50, a first lift actuator 52a, a second lift actuator 52b, a brake sensor 54, an scale/exit detection system 46, an Alternating Current (AC) power input 56, an AC power sensor 58, one or more control panels 42, an off-board network transceiver 60 having a signal strength detector 75, a nurse call cable interface 62, a plurality of siderail sensors 63, a location transceiver 64, a head of bed (HOB) angle sensor 69, a battery 71, and a battery monitor 73. Additionally, patient support apparatus 20 includes a first lift sensor 66a, a second lift sensor 66b, a cable sensor 68, display 70, and one or more controls 72 incorporated into one or more of the control panels 42. Still further, patient support apparatus 20 includes a mattress 38 having at least one mattress sensor 39 positioned therein. It will be understood by those skilled in the art that patient support apparatus 20 may be modified to include additional components not shown in FIG. 2, as well modified to include fewer components from what is shown in FIG. 2.

Controller 48 (FIG. 2) is constructed of any electrical component, or group of electrical components, that are capable of carrying out the functions described herein. In many embodiments, controller 48 is a conventional microcontroller, or group of conventional microcontrollers, although not all such embodiments need include a microcontroller. In general, controller 48 includes any one or more microprocessors, field programmable gate arrays, systems on a chip, volatile or nonvolatile memory, discrete circuitry, and/or other hardware, software, or firmware that is capable of carrying out the functions described herein, as would be known to one of ordinary skill in the art. Such components can be physically configured in any suitable manner, such as by mounting them to one or more circuit boards, or arranging them in other manners, whether combined into a single unit or distributed across multiple units as part of an embedded network. When implemented to include an embedded network, the embedded network may include multiple nodes that communicate using one or more of the following: a Controller Area Network (CAN); a Local Interconnect Network (LIN); an I-squared-C serial communications bus; a serial peripheral interface (SPI) communications bus; any of RS-232, RS-422, and/or RS-485 communication interfaces; a LonWorks network, and/or an Ethernet. The instructions followed by controller 48 in carrying out the functions described herein, as well as the data necessary for carrying out these functions, are stored in memory 50, and/or in one or more other memories accessible to the one or more microprocessors, microcontrollers, or other programmable components of controller 48. Memory 50 also includes a unique identifier 186 that uniquely identifies the particular patient support apparatus into which it is incorporated, such as, but not limited to, a serial number.

When controller 48 is implemented to communicate using an on-board Ethernet, the on-board Ethernet may be designed in accordance with any of the Ethernet-carrying patient support apparatuses disclosed in commonly assigned U.S. patent application Ser. No. 14/622,221 filed Feb. 13, 2015, by inventors Krishna Bhimavarapu et al. and entitled COMMUNICATION METHODS FOR PATIENT HANDLING DEVICES, the complete disclosure of which is incorporated herein by reference. In some embodiments, controller 48 may be implemented to include multiple nodes that communicate with each other utilizing different communication protocols. In such embodiments, controller 48 may be implemented in accordance with any of the embodiments disclosed in commonly assigned U.S. patent application Ser. No. 15/903,477 filed Feb. 23, 2018, by inventors Krishna Bhimavarapu et al. and entitled PATIENT CARE DEVICES WITH ON-BOARD NETWORK COMMUNICATION, the complete disclosure of which is incorporated herein by reference.

Controller 48 is shown in FIG. 2 as including a usage monitor 77. Usage monitor 77 monitors the usage of the various components of patient support apparatus 20 and determines if servicing of any of these components should be performed. In some embodiments, usage monitor 77 is carried out purely within controller 48 based upon inputs from the various components of the patient support apparatus 20. In other embodiments, usage monitor 77 may include one or more electrical circuits and/or devices separate from controller 48 that operate in conjunction with controller 48 to monitor the usage, and servicing of, the various components of patient support apparatus 20.

In at least one embodiment, usage monitor 77 includes a data logger that keeps track of the number of times each of the serviceable components (e.g. any component that moves, such as siderails 36, actuators 52, etc.) are moved, activated, or otherwise used. Once the number reaches a threshold—without having been serviced—controller 48 issues a notification and/or alert. The notification/alert may be sent to patient support apparatus server 86 and software application 110 for forwarding to display device 104 so that caregivers are apprised of the need for servicing one or more components of the patient support apparatus 20.

In some embodiments, usage monitor 77 is implemented to perform the functions of the data logger disclosed in commonly assigned U.S. Pat. No. 7,690,059 issued Apr. 6, 2017, to Lemire et al. and entitled HOSPITAL BED, the complete disclosure of which is incorporated herein by reference. In some embodiments, usage monitor 77 may alternatively, or additionally, perform any of the functions of the diagnostic and control system disclosed in the aforementioned '059 patent. In some embodiments, usage monitor 77 may perform, either alone or in addition to other structures, any of the servicing, monitoring, and/or event detection functions of the equipment management system disclosed in commonly assigned PCT patent publication WO 2018/013666 filed Jul. 12, 2017, by inventors David Becker et al. and entitled EQUIPMENT MANAGEMENT SYSTEM, the complete disclosure of which is incorporated herein by reference. Usage monitor 77 may alternatively or additionally perform still other usage and/or diagnostic monitoring.

First and second lift actuators 52a and 52b (FIG. 2) are components of lifts 26 and are configured to raise and lower litter frame 28 with respect to base 22. A first one of lift actuators 52a powers a first one of the lifts 26 positioned adjacent a head end of patient support apparatus 20 and a second one of lift actuators 52b powers a second one of the lifts 26 positioned adjacent a foot end of patient support apparatus 20. Lift actuators 52a and 52b may be conventional linear actuators having electric motors therein that, when driven, expand or contract the length of the linear actuator, thereby moving the litter frame upward or downward and changing its height H (FIG. 1) relative to the floor.

Each lift actuator 52a and 52b includes a corresponding lift sensor 66a and 66b, respectively. Each of the sensors 66a, 66b detects a position and/or angle of its associated actuator 52a, 52b and feeds the sensed position/angle to controller 48. Controller 48 uses the outputs from sensors 66 as inputs into a closed-loop feedback system for controlling the motion of the actuators 52a, 52b and the litter deck. Controller 48 also uses the outputs from sensors 66a, 66b to determine the height H of litter frame 28 above the floor. In some embodiments, actuators 52 are constructed in any of the same manners as the actuators 34 disclosed in commonly assigned U.S. patent application Ser. No. 15/449,277 filed Mar. 3, 2017, by inventors Anish Paul et al. and entitled PATIENT SUPPORT APPARATUS WITH ACTUATOR FEEDBACK, the complete disclosure of which is incorporated herein by reference. In such embodiments, sensors 66a and 66b may be constructed to include any of the encoders and/or switch sensors disclosed in the aforementioned '277 application.

Scale/exit detection system 46 is configured to determine a weight of a patient positioned on support deck 30 and/or when the patient is moving and is likely to exit patient support apparatus 20. The particular structural details of the exit detection system can vary widely. In some embodiments, scale/exit detection system 46 includes a plurality of load cells arranged to detect the weight exerted on litter frame 28. By summing the outputs from each of the load cells, the total weight of the patient is determined (after subtracting the tare weight). Further, by using the known position of each of the load cells, controller 48 determines a center of gravity of the patient and monitors the center of gravity for movement beyond one or more thresholds. One method of computing the patient's center of gravity from the output of such load cells is described in more detail in commonly assigned U.S. Pat. No. 5,276,432 issued to Travis and entitled PATIENT EXIT DETECTION MECHANISM FOR HOSPITAL BED, the complete disclosure of which is incorporated herein by reference. Other methods by which scale/exit detection system 46 may be implemented in order to determine when a patient is likely to exit from patient support apparatus 20 are disclosed in commonly assigned U.S. patent application Ser. No. 17/318,476 filed May 12, 2021, by inventors Sujay Sukumaran et al. and entitled PATIENT SUPPORT APPARATUS WITH EXIT DETECTION MODES OF OPERATION, the complete disclosure of which is incorporated herein by reference. Still other methods of detecting when a patient has exited, or is about to exit, from patient support apparatus 20 may be implemented by scale/exit detection system 46.

Scale/exit detection system 46 may also implement one or more other methods for determining a patient's weight and/or the weight of non-patient objects supported on litter frame 28, such as any of the methods and/or structures that are disclosed in commonly assigned U.S. patent application Ser. No. 14/776,842, filed Sep. 15, 2015, by inventors Michael Hayes et al. and entitled PATIENT SUPPORT APPARATUS WITH PATIENT INFORMATION SENSORS, and commonly assigned U.S. patent application Ser. No. 14/873,734 filed Oct. 2, 2015, by inventors Marko Kostic et al. and entitled PATIENT SUPPORT APPARATUSES WITH MOTION MONITORING, the complete disclosures of both of which are incorporated herein by reference. Scale/exit detection system 46 may utilize still other methods and/or structures for determining a patient's weight.

Mattress 38 is an inflatable mattress in many embodiments. In some embodiments, mattress 38 includes its own internal controller (not shown) that controls the inflation and deflation of various bladders contained within mattress under the instructions of controller 48. It will therefore be understood that the control of mattress 38 carried out by controller 48 may include both the direct control over the blower(s), pump(s), valve(s), and other components of mattress 38, or an indirect control over on onboard mattress controller that itself carries out the direct controls of the blower(s), pump(s), valve(s), and other components of mattress 38. In either situation, controller 48 may communicate with mattress 38 using a serial cable, or other cable, that extends between patient support apparatus 20 and mattress 38. In at least one alternative embodiment, the communication between patient support apparatus 20 and mattress 38 may be carried out wirelessly, such as in any of the manners disclosed in commonly assigned U.S. Pat. No. 9,289,336 issued to Lambarth et al. and entitled PATIENT SUPPORT WITH ENERGY TRANSFER, the complete disclosure of which is incorporated herein by reference. Other manners for wireless communication may, of course, be used.

In some embodiments, mattress 38 is constructed in accordance with any of the mattresses disclosed in commonly assigned U.S. Pat. No. 9,468,307 issued to Lafleche et al. and entitled INFLATABLE MATTRESS AND CONTROL METHODS, the complete disclosure of which is incorporated herein by reference. In other embodiments, mattress 38 is constructed in accordance with any of the mattresses disclosed in commonly assigned U.S. Pat. No. 8,413,271 issued to Blanchard and entitled PATIENT SUPPORT APPARATUS, the complete disclosure of which is also incorporated herein by reference. Other mattresses may also be used. Regardless of the specific construction of mattress 38, mattress 38 may be configured to carry out one or more different therapy procedures for the patient supported thereon. Such therapy procedures may include, but are not limited to, any of the following: rotation, percussion, vibration, maximum inflation, and turn assistance. Mattress 38 may also be able to be inflated to different states, thereby changing the distribution of pressure on the patient's skin while supported thereon. These various therapies and/or states are often used in order to reduce the likelihood of a patient developing a bed sore or exacerbating an already existing bed sore. Caregiver assistance system 106 is adapted to remotely communicate information about any of these therapies to software application 110 of patient support apparatus server 86, which is configured to forward this information to one or more display devices 104 that enable remotely positioned personnel to see this therapy information.

Controller 48 communicates with network transceiver 60 (FIG. 2) which, in at least one embodiment, is a Wi-Fi radio communication module configured to wirelessly communicate with wireless access points 76 of local area network 74. In such embodiments, network transceiver 60 may operate in accordance with any of the various IEEE 802.11 standards (e.g. 802.11b, 802.11n, 802.11g, 802.11ac, 802.11ah, etc.). In other embodiments, network transceiver 60 may include, either additionally or in lieu of the Wi-Fi radio and communication module, a wired port for connecting a network wire to patient support apparatus 20. In some such embodiments, the wired port accepts a category 5e cable (Cat-5e), a category 6 or 6a (Cat-6 or Cat-6a), a category 7 (Cat-7) cable, or some similar network cable, and transceiver 60 is an Ethernet transceiver. In still other embodiments, network transceiver 60 may be constructed to include the functionality of the communication modules 56 disclosed in commonly assigned U.S. patent application Ser. No. 15/831,466 filed Dec. 5, 2017, by inventor Michael Hayes et al. and entitled NETWORK COMMUNICATION FOR PATIENT SUPPORT APPARATUSES, the complete disclosure of which is incorporated herein by reference.

Regardless of the specific structure included with network transceiver 60, controller 48 is able to communicate with the local area network 74 (FIG. 2) of a healthcare facility in which the patient support apparatus is positioned. When network transceiver 60 is a wireless transceiver, it communicates with local area network 74 via one or more wireless access points 76. When network transceiver 60 is a wired transceiver, it communicates directly via a cable coupled between patient support apparatus 20 and a network outlet positioned within the room of the healthcare facility in which patient support apparatus 20 is positioned. As will be discussed in greater detail below with respect to FIG. 4, local area network 74 includes a plurality of servers that are utilized in different manners by the caregiver assistance system 106 disclosed herein, and patient support apparatus 20 communicates with one or more of those servers via transceiver 60 as part of the caregiver assistance system.

When network transceiver 60 is implemented as a wireless transceiver, it may include a signal strength detector 75 that detects the signal strength of the wireless signals it is receiving from the wireless access point 76 with which it is in communication. In some embodiments, such as ones in which network transceiver 60 is a WiFi transceiver, the signal strength detector 75 may be part of the conventional WiFi circuitry that determines the Received Signal Strength Indicator (RSSI), in which case the signal strength may be measured as an RSSI value. In other embodiments, the signal strength may be measured as an actual value in milliwatts (or other units), and signal strength detector 75 may be comprised of any conventional circuitry configured to measure the signal strength in this manner. In still other embodiments, signal strength detector 75 and/or patient support apparatus 20 may have any of the spectrum analysis functionality built into either or both of them that is disclosed in commonly assigned U.S. patent application Ser. No. 15/236,452 filed Sep. 29, 2016, by inventors Krishna Bhimavarapu et al. and entitled PERSON SUPPORT APPARATUSES WITH COMMUNICATION CHANNEL MONITORING, the complete disclosure of which is incorporated herein by reference.

Regardless of whether signal strength detector 75 measures signals using a relative RSSI value or an actual milliwatt value (e.g. −dBm), signal strength detector 75 is configured to forward its results to controller 48 which then displays the value on display 70 and also forwards the value to patient support apparatus server 86, which executes software application 110. Software application 110 may forward this signal strength value to one or more display devices 104 for display thereon.

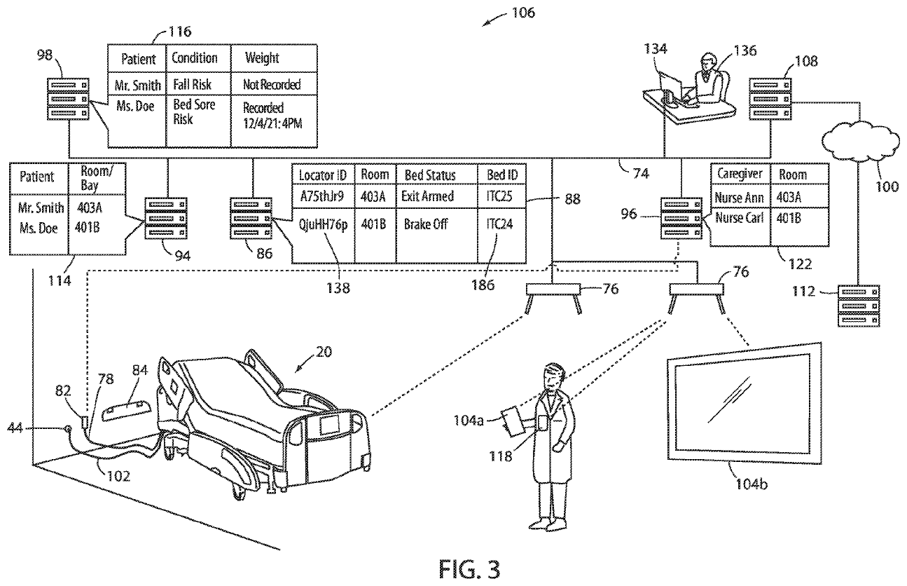

Nurse call cable interface 62 is an interface adapted to couple to one end of a nurse call cable 78 (FIG. 3). The other end of the nurse call cable 78 couples to a nurse call outlet 82 (FIG. 3) that is typically built into each headwall of each of the patient rooms within a healthcare facility. In many embodiments, nurse call outlet 82 is a 37 pin outlet that cable 78 couples to, thereby enabling patient support apparatus 20 to communicate directly with a conventional nurse call system. In some embodiments, nurse call cable interface 62 is constructed in accordance with any of the cable interfaces 92 disclosed in commonly assigned U.S. patent application Ser. No. 15/945,437 filed Apr. 4, 2018, by inventors Krishna Bhimavarapu et al. and entitled PATIENT SUPPORT APPARATUSES WITH RECONFIGURABLE COMMUNICATION, the complete disclosure of which is incorporated herein by reference.

In other embodiments, nurse call cable interface 62 may be replaced with a wireless nurse call communication system that wirelessly communicates with nurse call outlet 82. For example, in some embodiments, nurse call cable interface 62 is replaced with a radio module, such as the radio module 60 disclosed in commonly assigned U.S. patent application Ser. No. 14/819,844 filed Aug. 6, 2015, by inventors Krishna Bhimavarapu et al. and entitled PATIENT SUPPORT APPARATUSES WITH WIRELESS HEADWALL COMMUNICATION, the complete disclosure of which is incorporated herein by reference. In such wireless headwall embodiments, a headwall module, such as headwall module 38 disclosed in the aforementioned '844 application, is included and coupled to nurse call outlet 82. Such a headwall module may replace and/or supplement the functions of location beacon 84, described below. Still other types of wireless communication between the patient support apparatus and nurse call outlet 82 may be implemented.

Regardless of whether nurse call interface 62 uses a wired cable connection to a nurse call outlet 82 on the headwall of the hospital room or it uses a wireless connection, nurse call interface 62 may also, or alternatively, perform any of the functions of the nurse call interfaces disclosed in commonly assigned U.S. patent application Ser. No. 62/833,943 filed Apr. 15, 2019, by inventors Alexander Bodurka et al. and entitled PATIENT SUPPORT APPARATUSES WITH NURSE CALL AUDIO MANAGEMENT, the complete disclosure of which is also incorporated herein by reference.

Siderail sensors 63, which may be conventional siderail sensors, are configured to detect when the siderails 36 are in a raised or lowered position. In most embodiments, a single siderail sensor 63 is included for each of the siderails 36. Therefore, in the embodiment of FIG. 1, patient support apparatus 20 includes four siderail sensors 63, one for detecting the position of each of the four siderails 36. In alternative embodiments, more than one siderail sensor 63 may be included for each siderail 36, such as a first siderail sensor 63 that detects when the siderail is raised and/or locked in its raised position, and a second siderail sensor 63 that detects when the siderail 36 is in its lowered position, and/or locked in its lowered position. In general, any switch or other type of sensor that is able to detect when the respective siderail 36 is in its raised and/or locked orientation can be used with patient support apparatus 20. The outputs of siderail sensors 63 are fed to controller 48 and are periodically sent to software application 110 of patient support apparatus server 86 as part of the patient support apparatus status updates that are discussed in greater detail below.

Location transceiver 64 (FIG. 2) is adapted to detect a wireless signal emitted from a nearby location beacon 84 (FIG. 3) that is positioned at a fixed and known location within the healthcare facility. Although FIG. 3 only illustrates a single one of these location beacons 84, it will be understood that a particular healthcare facility includes many of these location beacons 84 mounted throughout the healthcare facility. Each location beacon 84 includes a wireless short range transmitter (not shown) that broadcasts a wireless, short range signal containing a unique identifier. The short range signal, in some embodiments, is broadcast via an infrared transmitter and is only detectable by receivers (e.g. location transceivers 64) that are positioned within several feet of the location beacon 84. Consequently, location transceivers 64, which are adapted to detect the signals transmitted from location beacons 84, are only able to detect these signals when patient support apparatuses 20 are positioned adjacent (e.g. within several feet) of one of these location beacons 84. If/when location transceiver 64 is able to detect the unique signal from a particular location beacon 84, the corresponding patient support apparatus 20 can therefore be concluded to be currently positioned adjacent that particular location beacon 84. This allows the current location of the patient support apparatus 20 to be identified. In some healthcare facilities, one or more of the patient rooms may not be completely private rooms, but instead may be shared with one or more other patients. In such situations, it is typical to mount two or more location beacons 84 within such a room-one on the headwall at the bay where the first patient support apparatus 20 normally resides and the other on the headwall at the bay where the second patient support apparatus 20 normally resides (and still more if the room is shared by more than two patients).

When location transceiver 64 receives a signal from an adjacent location beacon 84, controller 48 forwards the received signal, including the unique ID of the beacon 84, to software application 110 of patient support apparatus server 86 (FIG. 2). Software application 110 includes and/or utilizes a table 88 (FIG. 3) that correlates beacon IDs to locations (e.g. rooms) within the healthcare facility. Software application 110 is thereby able to determine the location of each patient support apparatus 20 within the healthcare facility (at least all of those that are positioned adjacent a location beacon 84).

In some embodiments, location beacons 84 (FIG. 2) function both as locators and as wireless links to the nurse call outlet 82 integrated into the adjacent headwall. When equipped with this dual function, patient support apparatuses 20 may omit the nurse call cable interface 62, yet still be able to communicate with the nurse call system. In the illustrated embodiment of FIG. 3, however, patient support apparatus 20 includes a nurse call cable 78 that communicatively couples the patient support apparatus 20 to nurse call outlet 82, thereby enabling the patient support apparatus 20 to communicate directly with the nurse call system. Further details about the function of location beacons 84, whether operating solely as locators or both as locators and wireless portals to the nurse call system outlets 82, may be found in any of the following commonly assigned U.S. patent references: U.S. Pat. No. 8,102,254 issued Jan. 24, 2012 to Becker et al. and entitled LOCATION DETECTION SYSTEM FOR A PATIENT HANDLING DEVICE; patent application Ser. No. 14/819,844 filed Aug. 6, 2015, by inventors Krishna Bhimavarapu et al. and entitled PATIENT SUPPORT APPARATUSES WITH WIRELESS HEADWALL COMMUNICATION; patent application Ser. No. 62/600,000 filed Dec. 18, 2017, by inventor Alex Bodurka, and entitled SMART HOSPITAL HEADWALL SYSTEM; and patent application Ser. No. 62/598,787 filed Dec. 14, 2017, by inventors Alex Bodurka et al. and entitled HOSPITAL HEADWALL COMMUNICATION SYSTEM, the complete disclosures of all of which are incorporated herein by reference.

Location beacon 84 also includes, in at least some embodiments, a beacon battery 79 and a beacon battery monitor 81 (FIG. 2). Beacon battery 79 provide electrical power to location beacon 84, either exclusively or, in at least some embodiments, when location beacon 84 is unplugged, or electrical power is otherwise unavailable from an electrical outlet. Beacon battery monitor 81 monitors the charge state of beacon battery 79 and reports measurements of this charge to patient support apparatus 20. That is, the measurements taken by beacon battery monitor 81 are forwarded wirelessly by locator beacon 84 to patient support apparatus 20 via the built-in transmitter of location beacon 84. These measurements are received by location transceiver 64 onboard patient support apparatus 20 and forwarded to controller 48. Controller 48 then displays these measurements on display 70 and/or forwards them to software application 110 via network transceiver 60. Software application 110 may forward these battery charge measurements to one or more display devices 104.

In some embodiments, beacon battery monitor 81 may monitor one or more additional factors regarding beacon battery 79, such as, but not limited to, the overall health of beacon battery 79. Such overall health may be measured in terms of the charge capacity of the battery, the number of times the battery has been recharged, the rate at which the battery discharges, the rate at which the battery re-charges, and/or in other manners. In some embodiments, beacon battery monitor 81 may be implemented in the same manner as, and/or configured to monitor and measure any one or more of the same battery parameters as, the battery monitors disclosed in commonly assigned U.S. patent publication 2016/0331614 published Nov. 17, 2016, and filed by inventors Aaron Furman et al. and entitled BATTERY MANAGEMENT FOR PATIENT SUPPORT APPARATUSES, the complete disclosure of which is incorporated herein by reference.

In some embodiments, locator beacon 84 may be incorporated into a wireless headwall module that communicates with patient support apparatus 20 over multiple communication channels. In such embodiments, the first communication channel between location beacon 84 and patient support apparatus 20 may be a short range channel (e.g. infrared) and the second one may be a longer range channel (e.g. Bluetooth). In such embodiments, the transmission of the data from beacon battery monitor 81 to patient support apparatus 20, as well as the transmission of the location identifier of locator beacon 84 to patient support apparatus 20, may occur over either or both of the two communication channels.

Controller 48 of patient support apparatus 20 (FIG. 2) communicates with AC power sensor 58, which informs controller 48 whether or not an AC power cable 102 (FIG. 3) is coupled between patient support apparatus 20 and a conventional alternative current (AC) power outlet 44. In other words, AC power sensor 58 lets controller 48 know whether patient support apparatus 20 is receiving electrical power from an off-board power supply (e.g. power outlet 44). In some cases, patient support apparatus 20 includes one or more batteries that are able to power patient support apparatus 20, including controller 48, when patient support apparatus 20 is not coupled to a source of electrical power. As will be discussed more below, the status of the AC power cord 102 (e.g. whether patient support apparatus 20 is operating on battery power or on power from an AC outlet) is communicated from AC power sensor 58 to controller 48, which then forwards that status via network transceiver 60 to software application 110 of patient support apparatus server 86.

Controller 48 also communicates with brake sensor 54 (FIG. 2). Brake sensor 54 informs controller 48 whether or not a brake has been applied on patient support apparatus 20. When the brake is applied, one or more of wheels 24 are braked to resist rotation. When the brake is not applied, wheels 24 are free to rotate. As with the data from the AC power sensor 58, the data from the brake sensor 54 is forwarded by controller 48 to software application 110 of patient support apparatus server 86 via network transceiver 60. Software application 110 shares this information with caregivers via one or more of the display devices 104 that are in communication with server 86, as will be discussed in greater detail below.

Controller 48 also communicates with head of bed angle sensor 69. Head of bed angle sensor 69 measures the angular orientation of head section 40 of patient support apparatus 20, either with respect to horizontal or with respect to the general plane of litter frame 28. In some embodiments, head of bed angle sensor 69 is implemented as one or more accelerometers that are mounted to head section 40. In other embodiments, head of bed angle sensor 69 may be implemented as an encoder counter, or other type of counter, that monitors the extension and retraction of the actuator that pivots head section 40. Still other types of sensors may be used to measure the angle of head section 40.

Regardless of the specific type of sensor used for HOB sensor 69, HOB sensor 69 reports its readings to controller 48, which in tum displays them on display 70 and/or forwards them to software application 110 via network transceiver 60. Software application 110 may forward the HOB angle reading to one or more display devices 104 for display thereon, thereby enabling caregivers to remotely view the current angle of head section 40 of patient support apparatus 20.

In some embodiments, patient support apparatus 20 may include one or more batteries 71 that are used to provide power to patient support apparatus 20, or certain components thereof, when patient support apparatus 20 is not electrically coupled to a conventional electrical power outlet. For example, in some embodiments, patient support apparatus 20 may include a first battery (or first set of batteries) that are used for all functions on the bed except for powering an onboard propulsion system, and a second battery (or second set of batteries) that are used for powering the onboard propulsion system. One example of such a patient support apparatus 20 is disclosed in commonly assigned U.S. patent application Ser. No. 62/823,324 filed Mar. 25, 2019, by inventors Zane Shami et al. and entitled PATIENT CARE SYSTEM WITH POWER MANAGEMENT, the complete disclosure of which is incorporated herein by reference. In other embodiments, patient support apparatus 20 includes only a single battery 71 (or a single set of batteries 71) that are used for powering all of the electrical functions of patient support apparatus 20. In many instances, whether one or more batteries 71 are included, such batteries 71 are typically rechargeable batteries 71.

In the embodiment shown in FIG. 2, patient support apparatus 20 further includes a battery monitor 73 that is adapted to monitor the charge state (and/or other parameters) of battery 71, or each of the batteries 71 (if there are more than one) of patient support apparatus 20. Battery monitor 73, in addition to monitoring the charge state of one or more batteries 71, may also monitor any of the same parameters of batteries 71 that beacon battery monitor 81 may monitor with respect to beacon battery 79, as discussed above. To that end, battery monitor 73 may be implemented in any of the same manners as, and/or perform any of the same functions as, the battery monitors disclosed in commonly assigned U.S. patent publication 2016/0331614 published Nov. 17, 2016, and filed by inventors Aaron Furman et al. and entitled BATTERY MANAGEMENT FOR PATIENT SUPPORT APPARATUSES, the complete disclosure of which has been incorporated herein by reference.

Regardless of which specific criteria battery monitor 73 monitors for one or more of the batteries 71 of patient support apparatus 20, battery monitor 73 reports the results to controller 48. Controller 48, in turn, displays one or more of the results on display 70 and/or forwards one or more of the results to software application 110. Software application 110 then forwards these results to one or more mobile display devices 104a, as discussed in more detail below. The display devices 104 include a display that is adapted to display information about the monitored state(s) of battery(ies) 71. If patient support apparatus 20 contains more than one battery, controller 48 forwards the monitored data for each battery (or set of batteries) to software application 110, which in tum forwards this data to one or more display devices 104. The recipient display device 104 may display the received data separately for each set of batteries. In this manner, caregivers who are remote from patient support apparatus 20 are able to review the status of each of the batteries onboard patient support apparatus 20.

Each of the control panels 42 includes one or more controls 72 that are used to control various functions of the patient support apparatus 20 (FIG. 2). For example, one or more of the control panels 42 includes a motion control 72 for controlling movement of the lift actuators 52a and 52b. Additional controls 72 may be provided for activating and deactivating the brake for wheels 24, arming and disarming exit detection function of scale/exit detection system 46, taking a weight reading of the patient using the scale function of scale/exit detection system 46, activating and deactivating a propulsion system (if included), pivoting the angle of head section 40 (and/or other sections of support deck 30) and communicating with one or more servers on local area network 74. It will be understood that in some embodiments, one or more of controls 72 may be integrated into a touchscreen display, such as display 70. In such embodiments, one or more of the controls may only appear when the user navigates to specific screens displayed on the touchscreen.

Patient support apparatus 20 communicates with the software application 110 of patient support apparatus server 86 via local area network 74 (FIG. 2). Software application 110 is adapted to assist the caregivers in performing a plurality of tasks. In general, software application 110 is adapted to assist the caregivers in ensuring that the patient support apparatuses 20 are maintained in a desirable state, to assist the caregivers in alerting them of undesired conditions associated with their patients and/or patient support apparatuses 20, and/or to assist in other ways.

In order to carry out its functions, software application 110 may include, or utilize, a set of local rules (local to a particular healthcare facility, or portion of a healthcare facility), a data repository, a communication interface, and/or a web Application Programming Interface. The set of local rules may be defined prior to the installation of software application 110 within a particular healthcare facility, and/or it may be modifiable by authorized personnel after installation within the healthcare facility. Such modifications are made by way of one or more computers 134 that are in communication with local area network 74 (FIG. 3) and that act as user interfaces for software application 110. Thus, an authorized individual 136 (FIG. 3) may utilize computer 134 to communicate with software application 110 and add, delete, or modify one or more of the local rules.

The local rules may include, but are not limited to, the following: rules indicating what state patient support apparatuses 20 are to be placed in; rules specifying who is to be notified, and when, if a patient support apparatuses is not placed in the desired state and/or is moved out of the desired state; rules specifying how such notifications are to be communicated (e.g. email, phone call, texts, etc.); rules specifying what personnel within the healthcare facility are authorized to view what data using software application 110. As will be discussed in greater detail below, the rules defining desired states of the patient support apparatuses 20 may be modified by authorized individuals 136 to vary based upon one or more factors. For example, these rules may be modified for different wings of the healthcare facility, different units of the healthcare facility, different times of day and/or different shifts, different models of patient support apparatuses, different patient health conditions, different patient treatments, different data stored in an EMR server 98, etc.

The local rules may also include additional administrative data that is stored on patient support apparatus server 86, or stored in a memory otherwise accessible to software application 110. Such administrative data includes, but is not limited to, the IP address, or other network address, of each of the servers with which software application 110 is to communicate (e.g. EMR server 98, an ADT server 94, a nurse call server 96, and/or other servers), and/or the IP addresses or other configuration data necessary for software application 110 to communicate with one or more middleware software applications that act as gateways to one or more of these servers. The administrative data also may also include the email addresses, passwords, phone numbers, caregiver badge IDs, user names, access levels, and other information about those hospital personnel who have been authorized to use software application 110. The email address and/or phone numbers are used in some embodiments of software application 110 that are configured to automatically send alerts to one or more caregiver tags and/or to one or more display devices 104.

The data depository that is part of, and/or that software application 110 has access to, stores data that is received by software application 110 during the course of its operation. This data includes patient support apparatus status conditions sent from patient support apparatuses 20 and notification data (e.g. when alerts occurred, causes, remedies, notifications, etc.). The data repository may also store room-to-bed and room bay-to-bed associations that correlate specific patient support apparatuses 20 to specific rooms and/or bays. In some embodiments, it may temporarily also store room-to-patient associations, bed-to-patient associations, caregiver-to-patient associations, caregiver-to-room associations, and/or caregiver-to-patient support apparatus associations.

The communication interface used by software application 110 controls the communications between software application 110 and the display devices 104 with which it is in communication. The communication interface may also control the communications between software application 110 and the servers with which it is in communication. All of these communications, in at least one embodiment, are carried out using conventional Internet packet routing. That is, patient support apparatuses 20 send data in packets that have an IP address corresponding to patient support apparatus server 86, and server 86 sends message packets back to patient support apparatuses 20 that include an IP address corresponding to the particular patient support apparatus(es) 20 to which the messages are intended. In some embodiments, each patient support apparatus 20 includes a static IP address that is stored on the patient support apparatus 20, while in other embodiments, the patient support apparatuses 20 consult a local Dynamic Host Configuration Protocol (DHCP) server (not shown) on local area network 74 and the DHCP server assigns a network address to the patient support apparatuses 20.

When communicating with other servers within the healthcare facility, the communication interface of software application 110 may utilize different communication protocols, such as, but not limited to, Link Layer Protocol (LLP), Hyper-Text Transfer Protocol Secure (HTTPS), and/or Simple Mail Transfer Protocol (SMTP), etc. In order to facilitate the communication between patient support apparatus server 86 and the other servers of local area network 74, the communication interface may utilize a conventional interface engine, such as, but not limited to, the Redox cloud platform that is commercially available from Redox, Inc. of Madison, Wisconsin. Alternatively, or additionally, the communication interface may utilize a conventional iGUANA interface engine (HL-7 or otherwise) available from INTERFACEWARE, Inc. of Toronto, Ontario. Such interfaces allow software application 110 to communicate with different types and/or brands of Electronic Health Record (EHR) systems, such as, but not limited to, those marketed by Cerner corporation, Epic Corporation, Allscripts, etc.

The web API that may be used in some embodiments of software application 110 provides a portal for authorized devices, software applications, and/or servers to access the data of software application 110. In some embodiments, display devices 104 communicate with software application 110 via the web API by using a web browser built into the display devices 104 that accesses one or more Uniform Resource Locators (URLs) that direct the web browser to software application 110. The web API, in some embodiments, uses JavaScript Object Notation (JSON) to communicate with the web browsers of the display devices 104. In other embodiments, the web API use Extensible Markup Language (XML) to communicate with the web browsers of the display devices 104. Still other types of communication may be used.

In some embodiments, the web API may be configured to communicate with the display devices 104 using the conventional GET, POST, DELETE, and UPDATE verbs of the Hyper-Text Transfer Protocol (HTTP). These are used for providing RESTful service (i.e. Representational State Transfers) between web API and the display devices 104. For those aspects of software application 110 that utilize two way interactive communication, conventional web socket protocols (e.g. IETF RFC 6455, or the WebSocket API in Web IDL (Interface Description Language) that is standardized by the World Wide Web Consortium (W3C) may be used for communication between the web API and the display devices 104. Alternatively, or additionally, conventional pull and push requests may be used for this communication, as well as, but not limited to, server-sent events and/or long polling. Still other communication techniques may be used. In some embodiments, such communications are encrypted such that at least those messages containing patient data are secured against interception. Such encryption takes place, in at least one embodiment, as part of a RESTulf Web service (RWS).

In general, software application 110 performs the following functions: gathers data from patient support apparatuses 20 about their current states; communicates the patient support apparatus data to display devices 104 that are remote from patient support apparatus server 86; causes the display devices 104 to display any undesired patient support apparatus conditions thereon; causes the display devices 104 to display reminders and/or alerts on their displays to assist caregivers in performing their tasks; communicates alerts to the caregivers if the patient support apparatus status conditions indicate the patient support apparatus 20 is not in a desired state or if a timer associated with the patient or the patient support apparatus 20 has expired; and/or performs other functions. In some embodiments, software application 110 may be configured to perform any one or more of the functions and/or algorithms performed by the caregiver assistance system disclosed in commonly assigned PCT patent application serial number PCT/US2021/033408, filed May 20, 2021, by applicant Stryker Corporation and entitled CAREGIVER ASSISTANCE SYSTEM, the complete disclosure of which is incorporated herein by reference.

Patient support apparatus 20 is shown in FIG. 2 positioned in a room 92 of a representative example of a healthcare facility. FIG. 2 also depicts patient support apparatus 20 in communication with local area network 74 of the healthcare facility. It will be understood that the precise structure and contents of the local area network 74 will vary from healthcare facility to healthcare facility. FIG. 3 illustrates in greater detail the contents of a common hospital's local area network 74, along with patient support apparatus server 86 and several display devices 104.

As shown in FIG. 3, local area network 74 includes a plurality of servers, including a conventional Admission, Discharge, and Tracking (ADT) server 94, a conventional nurse call server 96, a conventional Electronic Medical Records server 98, and a plurality of conventional wireless access points 76. Local area network 74 also includes patient support apparatus server 86 that, together with one or more patient support apparatuses 20 and one or more display devices 104, implement one embodiment of the caregiver assistance system 106 according to the present disclosure. Still further, network 74 includes a conventional Internet gateway 108 that couples local area network 74 to the Internet 100, thereby enabling the servers and/or patient support apparatuses 20 to communicate with computers outside of the healthcare facility, such as, but not limited to, a geographically remote server 112. In some embodiments, all or some of the functions of software application 110 of patient support apparatus server 86 are carried out by geographically remote server 112, while in other embodiments software application 110 of patient support apparatus server 86 is configured to implement all or some of its functions without accessing geographically remote server 112.

ADT server 94, which may be a conventional server, stores patient information, including the identity of patients and the corresponding rooms 92 and/or bays within rooms to which the patients are assigned. That is, ADT server 94 includes a patient-room assignment table 114 (FIG. 3), or functional equivalent to such a table. The patient-room assignment table 114 correlates rooms, as well as bays within multi-patient rooms, to the names of individual patients within the healthcare facility. The patient's names are entered into the ADT server 94 by one or more healthcare facility staff whenever a patient checks into the healthcare facility and the patient is assigned to a particular room within the healthcare facility. If and/or when a patient is transferred to a different room and/or discharged from the healthcare facility, the staff of the healthcare facility update ADT server 94. ADT server 94 therefore maintains an up-to-date table 114 that correlates patient names with their assigned rooms. ADT server 94 may be a conventional server marketed by Cerner Corporation of North Kansas City, Missouri; EPIC Systems of Madison, Wisconsin; Allscripts Healthcare Solutions, Inc. of Chicago, Illinois; and/or by other companies. Still other types of ADT servers 94 may, of course, be used. In some embodiments, ADT server 94 and/or a portion of its functions may be integrated into, or combined with, those of EMR server 98.

EMR server 98 (FIG. 3) stores the medical records of individual patients. Such patient records identify a patient by name and the medical information associated with that patient. Such medical information may include all of the medical information generated from the patient's current stay in the healthcare facility as well as medical information from previous visits. EMR table 116 shows an abbreviated example of three types of medical information entries that are commonly found within a patient's medical records: a fall risk entry indicating whether the patient is a fall risk, a bed sore risk entry indicating whether the patient is at risk for developing bed sores, and a weight record indicating one or more measurements of the patient's weight that were made while the patient is staying at the medical facility. Although FIG. 3 shows the data for the first two of these entries expressed as text, it will be understood that this data may be stored within a medical record in numeric format. For example, the fall risk data may be stored as a numeric value generated from a conventional fall risk assessment tool, such as, but not limited to, the Morse fall risk scale or the Hester-Davis fall risk scale. Similarly, the bed sore data may be stored as a numeric value generated from a conventional bed sore risk assessment tool, such as, but not limited to, the Braden scale. The weight data may include not only the patient's weight reading(s), but also the date and/or time at which these weight measurements were taken.

As noted, a typical EMR server 98 will include far more additional information in the medical records of each patient than what is shown in table 116 of FIG. 3. It will be understood that the term “EMR server,” as used herein, also includes Electronic Health Records servers, or EHR servers for short, and that the present disclosure does not distinguish between electronic medical records and electronic health records. EMR server 98 may be a conventional server marketed by Cerner Corporation of North Kansas City, Missouri; EPIC Systems of Madison, Wisconsin; Allscripts Healthcare Solutions, Inc. of Chicago, Illinois; and/or by other companies. Still other types of EMR servers 98 may, of course, be used.

Nurse call server 96 is shown in FIG. 3 to include a caregiver assignment table 122 that matches caregivers to specific rooms and/or bays within the healthcare facility. Although table 122 only shows caregivers assigned to a single room, it will be understood that each caregiver is typically assigned to multiple rooms. In some nurse call systems, caregivers are assigned to specific patients, rather than to specific rooms. Caregiver assistance system 106 is configured to work with both types of nurse call systems. Caregiver assistance system 106 is also adapted to work with healthcare facilities that utilize a separate caregiver assignment server (not shown), rather than nurse call server 96, to assign caregivers to rooms and/or patients. Nurse call server 96 may be a conventional server marketed by Rauland (now owned by Ametek, Inc. of Berwyn, Pennsylvania); by West-Com Nurse Call System, Inc. of Fairfield, California; and/or by other companies.

Regardless of whether caregiver assignment table 122 is stored within nurse call server 96 or some other server on network 74, nurse call server 96 is configured to communicate with caregivers and patients. That is, whenever a patient on a patient support apparatus 20 presses, or otherwise activates, a nurse call, the nurse call signals pass through nurse call cable 78 to nurse call outlet 82. Nurse call outlet 82 is coupled via wire to nurse call server 96 and/or to another structure of the nurse call system that then routes the call to the appropriate nurse. The nurse is thereby able to communicate with the patient from a remote location.

Local area network 74 may include additional structures not shown in FIG. 3, such as, but not limited to, one or more conventional work flow servers and/or charting servers that monitor and/or schedule patient-related tasks for particular caregivers, and/or one or more conventional communication servers that forward communications to particular individuals within the healthcare facility, such as via one or more portable devices (smart phones, pagers, beepers, laptops, etc.). The forwarded communications may include data and/or alerts that originate from patient support apparatuses 20 as well as data and/or alerts that originate from patient support apparatus server 86.

Wireless access points 76 are configured, in at least some embodiments, to operate in accordance with any one or more of the IEEE 802.11 standards (e.g. 802.11g, 802.11n, 802.11ah, etc.). As such, patient support apparatuses 20 and display devices 104 that are equipped with Wi-Fi capabilities, and that have the proper authorization credentials (e.g. password, SSID, etc.), can access local area network 74 and the servers hosted thereon. This allows patient support apparatus 20 to send messages to, and receive messages from, software application 110 of patient support apparatus server 86. This also allows display devices 104 to send messages to, and receive messages from, software application 100 110 of patient support apparatus server 86. As noted previously, alternatively, or additionally, patient support apparatuses 20 may include a wired port for coupling a wired cable (e.g. a Category 5, Category 5e, etc.) between the patient support apparatus 20 and one or more routers/gateways/switches, etc. of network 74, thereby allowing patient support apparatuses 20 to communicate via wired communications with software application 110 of server 86.

In still other embodiments, one or more of the patient support apparatuses 20 are equipped with alternative wireless transceivers enabling them to communicate directly with patient support apparatus server 86 via an antenna and transceiver that is directly coupled to server 86 and that is separate from LAN 74, thereby allowing patient support apparatuses 20 to bypass LAN 74 in their communications with server 86. One example of patient support apparatuses equipped to communicate directly with a server on a healthcare facility's local area network without utilizing the LAN is disclosed in commonly assigned U.S. patent application Ser. No. 15/831,466 filed Dec. 5, 2017, by inventors Michael Hayes and entitled NETWORK COMMUNICATION FOR PATIENT SUPPORT APPARATUSES, the complete disclosure of which is incorporated herein by reference. In some embodiments, patient support apparatuses 20 include communication modules, such as the communication modules 66 disclosed in the aforementioned '466 application, and server 86 is coupled directly to a receiver, such as the enterprise receiver 90 disclosed in the aforementioned '466 application. In such embodiments, patient support apparatuses 20 are able to both send and receive messages directly to and from server 86 without utilizing access points 76 or any of the hardware of network 74 (other than server 86).

Software application 110 of patient support apparatus server 86 is configured to construct a table 88 (FIG. 3), or a similar type of data structure, that determines in which specific rooms 92—and/or bays within the rooms of the healthcare facility—each of the patient support apparatuses 20 is currently located. Software application 110 determines these room and/or bay locations by using the known location of each locator beacon 84 within the healthcare facility (which may be determined via a surveying procedure during the installation of beacons 84) and the known beacon IDs 138 of each locator beacon 84. Each locator beacon 84 sends its unique beacon ID 138 to an adjacent patient support apparatus 20 when the patient support apparatus 20 is positioned within a close proximity of the patient support apparatus 20. The patient support apparatus 20, in turn, forwards this unique beacon ID 138, along with its unique patient support apparatus ID 186, to software application 110. Software application 110 then uses these two IDs, along with the known location of the locator beacons 84, to determine the location of each patient support apparatus 20.

Software application 110 also receives status conditions from each patient support apparatus 20. Such status conditions may include data from any of the various sensors onboard patient support apparatus 20. Each patient support apparatus 20 sends these status conditions to software application 110 with its corresponding unique patient support apparatus ID 186. Software application 110 is therefore able to correlate incoming patient support apparatus status conditions with specific patient support apparatuses 20 and specific locations within the healthcare facility. In other words, software application 110 is able to construct a data structure like table 88 of FIG. 3, which includes the room location and status conditions for each of the patient support apparatuses 20 within the healthcare facility (or within a portion of the healthcare facility).

Although not shown in table 88 of FIG. 3, software application 110 may also correlate the information of table 88 to one or more additional pieces of information, such as specific caregivers, specific patients, and/or other pieces of information. For example, software application 110 may determine which caregivers are associated with each of the patient support apparatuses 20 based on the caregiver-to-room assignment data it receives from nurse call server 96 (i.e. the data of table 122). By using this caregiver-to-room assignment data, software application 110 is able to determine which caregiver(s) are assigned to each of the patient support apparatuses 20. Further, software application 110 may determine which patients are associated with each of the patient support apparatuses 20 based on the patient-to-room assignment data it receives from ADT server 94 (i.e. the data of table 114). By using this patient-to-room assignment data, software application 110 is able to determine which patient is assigned to each of the patient support apparatuses 20. Further, by knowing which patient is assigned to each patient support apparatus 20, software application 110 is able to assign medical information received from EMR server 98 to each of the patient support apparatuses 20 (e.g. the patient assigned to a specific patient support apparatus 20 has an elevated fall risk, a lower bed sore risk, and was last weighed at such-and-such a time). In summary, software application is supplied with sufficient data to know the current status of each patient support apparatus 20, the room in which each patient support apparatus 20 is assigned, the caregiver assigned to that room and/or patient support apparatus 20, the patient assigned to each patient support apparatus 20, and the fall risk, bed sore risk, weight history, and/or other medical data of each patient.

In some embodiments, software application 110 is configured to determine patient-to-room, patient-to-bed, patient-to-bed-bay, patient-to-caregiver, caregiver-to-room, caregiver-to-patient-support-apparatus, and/or caregiver-to-bed-bay correlations in any of the manners disclosed in commonly assigned U.S. patent application Ser. No. 62/826,097, filed Mar. 29, 2019 by inventors Thomas Durlach et al. and entitled PATIENT CARE SYSTEM, the complete disclosure of which is incorporated herein by reference. In some embodiments, software application 110 may further be modified to carry out any of the staffing errors, and other error-notification functions, disclosed in the aforementioned '097 application.

Display devices 104 (FIG. 3) may come in a variety of different forms. As shown in FIG. 3, some display devices 104 are mobile display devices 104a intended to be carried by a user (e.g. caregiver) while other display devices 104 are stationary display devices 104b that generally remain in one location. Mobile display devices 104a may take on different forms, such as, but not limited to, smart phones, tablets, laptop computers, Computers on Wheels (COWs), and others. Stationary display devices 104b may also take on different forms, such as, but not limited to, smart televisions, displays, Personal Computers (PCs), and others. For purposes of the following written description, caregiver assistance system 106 will be described with reference to display devices 104 that communicate with software application 110 via a conventional web browser. It will be understood, however, that in other embodiments, some or all of the display devices 104 may be modified to execute a specialized or native software application that is downloaded to the display device 104 and that is tailored to be executed by the particular operating system of the display device (e.g. Android, IOS, Windows, etc.). The specialized software application is executed by the microcontroller(s) of the display device 104 and carries out the functions of caregiver assistance system 106 described herein.

In some embodiments, in order for a caregiver associated with a display device 104 to access caregiver assistance system 106, the caregiver utilizes the web-browsing application contained within the display device 104 to go to a particular web page, or other URL, associated with software application 110. Any conventional web-browsing software may be used for this purpose, including, but not limited to, Microsoft's Bing or Internet Explorer web browsers, Google's Chrome web browser, Apple's Safari web browser, Mozilla's Firefox web browser, etc. The particular URL accessed with the web browser may vary for different healthcare facilities and can be customized by authorized IT personnel at the healthcare facility. In some embodiments, a domain name may be associated with software application 110 that is resolved by a local DNS server to the IP address of patient support apparatus server 86 (e.g. www.caregiver-assistance-app.com). In other embodiments, display devices 104 may include their own native software applications that are programmed to interact with software application 110, thereby avoiding the usage of a web browser to access software application 110. Access to software application 110 may be achieved in other manners. As noted, the following description will focus primarily on using a conventional web browser onboard display devices 104 to access the caregiver assistance application, but it will be understood that display devices 104 may include their own software apps that are specifically tailored to interact with software application 110.

Software application 110 may be configured to require a user to enter a user name and/or password via the display device 104 before the user is able to access software application 110. After entering the appropriate information into a display device 104, the software application 110 is configured to instruct the display device 104 to display data regarding one or more patient support apparatuses 20 that are positioned within the healthcare facility. Such data may include a dashboard screen, such as the dashboard screen 120 of FIG. 4.

Dashboard screen 120 is particularly suited for being displayed on display devices 104 that have a relatively large display size, such as stationary display devices 104b (and not, for example, mobile display devices 104a that have a relatively small screen, such as smart phones or small computers). Dashboard screen 120 includes a plurality of room icons 124 (i.e. enclosures that are defined by rectangles having rounded corners). Each room icon 124 corresponds to a particular room and/or bay within an actual room of the healthcare facility in which caregiver assistance system 106 is installed. Thus, in the example shown in FIG. 4, there are thirty room icons 124. Each room icon 124 includes a header portion 126 that identifies the particular room in the healthcare facility to which the room icon 124 corresponds and a body portion 128 that, as will be discussed more below, may display information about the status of the patient support apparatus 20 positioned within that particular room.

As shown in FIG. 4, header portion 126 is color coded. That is, software application 110 in configured to instruct the display device 104 to display header portion 126 in different colors depending upon the fall risk of the patient assigned to that particular room. In the example dashboard screen 120 of FIG. 4, header portion 126 of room NW1 has a green background, which indicates that the patient in room NW1 has a low fall risk. In contrast, the patient assigned to room NW5 in the example of FIG. 4 has a medium fall risk, and software application 110 is configured to instruct display device 104 to display the header portion 126 for room NW5 with a blue background. In the example of FIG. 4, software application 110 instructs display device 104 to display header portion 126 with a yellow background for those patients having a high fall risk, and a gray background for those patients whose fall risk has not yet been determined. As shown by room NW2 of screen 120, software application 110 may instruct display device 104 to omit header portion 126 in those rooms for which no patient is assigned (or to use a white background that blends in with the white background of body portion 128).

In some embodiments, software application 110 is configured to allow the user to customize the precise colors that are displayed in header portion 126 for each of the different fall risks of the patients. In this manner, software application 110 can be configured to display colors that precisely match whatever color scheme the healthcare facility may already have in place for categorizing patients according to their fall risk. The color customization may allow the user to specify the Red, Green, and Blue (RGB) values of the background colors of header portions 126. In addition, the color customization of software application 110 may be configured to allow the user to customize the colors of the text that appears in the header portions 126 (in addition to the background colors), as well as the color of the icons, text, and/or background of body portions 128 of the room icons 124.

Software application 110 is configured to instruct the display devices 104 to display selected undesired patient support apparatus conditions in the body portions 128 of each room icon 124. In general, software application 110 instructs the display devices 104 to display data regarding any undesired conditions and/or undesired states of the patient support apparatus 20. More specifically, software application 110 instructs the display devices 104 to display a specific indicator 130 and/or a generic indicator 132. The specific indicator 130 specifically identifies the specific condition that is currently present on the patient support apparatus, while the generic indicator 132 only generally identifies one or more undesired conditions that are currently present on the patient support apparatus 20. Thus, a caregiver looking at dashboard screen 120 can discern from the specific indicators 130 what specific undesired conditions are present on each patient support apparatus 20. The caregiver cannot, however, discern what specific undesired conditions are present on each patient support apparatus 20, but instead can only discern the general fact that undesired conditions are present.

The distinction between the specific indicators 130 and the generic indicators 132 can be more easily understood with respect to several of the examples shown in FIG. 4. One of the specific indicators 130 shown therein is the specific indicator 130 of room NW7. Specific indicator 130 of room NW7 specifically identifies that a siderail is not raised on the patient support apparatus 20 currently positioned in room NW7. Thus, a viewer of dashboard screen 120 knows the specific undesired condition (siderail not raised) for the patient support apparatus of room NW7. Similarly, the specific indicator 130 for room NW8 indicates that the brake is not set on the patient support apparatus 20 of that room. Thus, a viewer of dashboard screen 120 know the specific undesired condition (brake not set) for the patient support apparatus of room NW8.

In contrast, room NW23 includes both a specific indicator 130 and a generic indicator 132. The specific indicator 130 indicates that the litter frame 28 of the patient support apparatus 20 in room NW23 is not lowered to its lowest (or otherwise desired) height. The generic indicator 132 of room NW23 indicates that there are two additional undesired conditions currently existing for the patient support apparatus 20 of room NW23. What those two additional undesired conditions are, however, is not indicated by general indicator 132. In some embodiments, where display device 104 is a touchscreen, the user is able to see what those two additional undesired conditions specifically are by touching on the room icon 124 corresponding to room NW23. However, in some embodiments, where the display device 104 is not a touchscreen, the user may not be able to determine what the specific undesired conditions are that correspond to the two additional undesired conditions of the generical undesired indicator 132 of room NW23 (or of any room having a generic indicator 132).

In at least one embodiment, software application 110 is configured to display a specific indicator 130 whenever there is only a single undesired condition currently present for a patient support apparatus 20, and to display a generic indicator 132 if there are more than one undesired conditions currently present for a patient support apparatus 20. Thus, for example, if a patient support apparatus 20 currently has its brake deactivated and its AC power cord not plugged in, software application 110 will display a specific indicator 130 for one of these undesired conditions and a generic indicator 132 (in this case the numeral 1) for the other undesired condition. Software application 110 chooses which undesired condition to display the specific indicator 130 for based on the priority assignments that the user is able to assign to each undesired condition, as will be discussed in greater detail below.

As another example, if a particular patient support apparatus 20 does not have its siderails raised, does not have its nurse call cable 78 plugged into outlet 82, and does not have its brake on, software application 110 is configured to instruct the display device to display a single specific indicator 130 for one of these undesired conditions and a generic indicator 132 for the other two undesired conditions. That is, software application 110, in at least one embodiment, only displays a single specific indicator 130 for each room icon 124. If there are more than one undesired conditions currently active for that patient support apparatus 20, software application 110 instructs the display device 104 to display a generic indicator 132 having a numeric value equal to the number of additional undesired conditions (i.e. one less than the total number of undesired conditions). If there is only a single undesired condition present for a patient support apparatus 20, software application 110 instructs the display device to display only a specific indicator 130 for that particular undesired condition and no generic indicator 132.

In some embodiments, the list of undesired conditions that are indicated on dashboard screen 120 is configurable by the user. That is, the user is able to define what conditions are to be monitored by software application 110 and reported on dashboard screen 120. In some embodiments, software application 110 is configured to be able to monitor any one or more of the following undesired conditions associated with a patient support apparatus 20: one or more siderails are not in a desired position (raised or lowered, or in an intermediate position); the brake is not set; the litter frame 28 is not at its lowest height (or within a desired range of heights); the exit detection system 46 is not armed; the exit detection system 46 is not armed with the desired sensitivity level; the angle of the head section (a.k.a. Fowler section) 40 of patient support apparatus 20 is not above a threshold value; a fall risk has not been performed for a patient; the AC power cord 102 is not plugged into an AC outlet 44; the nurse call cable 78 is not plugged into a nurse call outlet 82 (or is otherwise not in communication with the nurse call system); a wireless connection between patient support apparatus 20 and the nurse call outlet 82 has not been established; exit detection system 46 is currently detecting an exit alert condition; the patient is currently not in patient support apparatus 20; or still other undesired conditions.

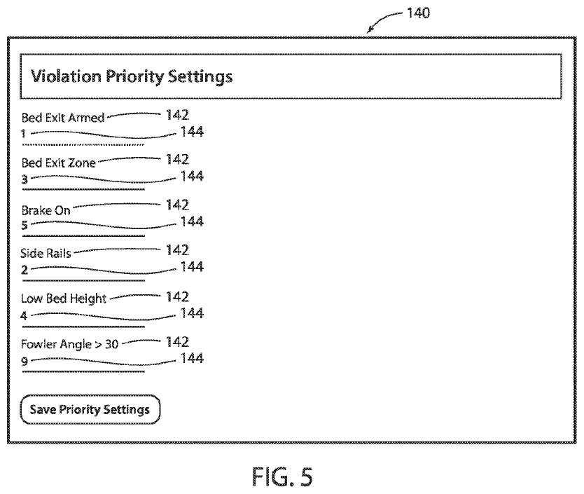

Software application 110 is configured to allow authorized users to assign priorities to each of these undesired conditions. These priority assignments determine which undesired condition will be displayed on dashboard screen 120 with a specific indicator 130 and which undesired condition(s) will be displayed on dashboard screen 120 with a generic indicator 132. One example of a prioritization screen 140 that may be displayed by software application 110 is shown in FIG. 5. Prioritization screen 140 allows a user to assig priority values to each of the undesired conditions that are monitored by software application 110.

As shown in FIG. 5, prioritization screen 140 displays a plurality of status conditions 142 and a plurality of priority assignments 144. For each status condition 142, a priority assignment 144 indicates a priority level for that corresponding status condition 142. Thus, for example, for the condition 142 of the exit detection system 46 not being armed, the priority assignment 144 has a value of 1. For the condition 142 of an incorrect sensitivity level being set for the exit detection system 46, the priority assignment 144 has a value of 3. For the condition 142 of the brake not being activated, the priority assignment 144 has a value of 5. The status conditions 142 for the siderails, low bed height, and Fowler angle have priority assignments 144 with values of 2, 4, and 9, respectively.

Each of the priority assignment 144 values may be changed by an authorized user of software application 110. To change the priority assignment values 144, an authorized user logs into the software application 110 (such as by using computer 134 (FIG. 3), or another computer coupled to network 74), enters their credentials, and navigates to the prioritization screen 140 (FIG. 5). Once at screen 140, the user is able to enter different numeric values for each of the priority assignments 144 using a conventional keyboard, keypad, mouse, touchscreen, etc. Software application 110 is configured to allow the user to assign whatever positive integer values the user desires to each priority assignment 144 so long as no two status conditions 142 have the same numeric priority value. That is, each priority assignment 144 must have a different value than the other priority assignment values.

Software application 110 uses the priority assignments 144 to determine which specific indicator 130 to display and which generic indicator 132 to display on dashboard screen 120 when multiple undesired conditions exist for a particular patient support apparatus 20. In general, if only a single undesired condition currently exists for a patient support apparatus 20, software application 110 displays the specific indicator 130 for that particular undesired condition, regardless of its priority assignment value 144. If more than one undesired condition currently exists for a particular patient support apparatus 20, software application 110 displays the specific indicator 130 for the status condition 142 having the highest priority assignment 144 (i.e. the highest numeric value for its priority assignment 144). Thus, in the example shown in FIG. 5, if a patient support apparatus 20 currently has the exit detection system 46 armed with the wrong sensitivity level and one of its siderails is in an undesired position, software application 110 will instruct the display device 104 to display the specific indicator 130 for the incorrect sensitivity level and a generic indicator 132 (shown with the number 1) for the siderail being in an undesired position. This is because, as shown in FIG. 5, the exit detection zone status condition 142 has a priority assignment of 3 and the siderail status condition 142 has a priority assignment of 2. Because 3 is greater than 2, the exit detection zone status condition 142 has a higher priority assignment 144 than the siderail condition, so software application 110 displays the specific indicator 130 for the exit detection zone undesired condition and the generic indicator 132 for the siderail undesired condition.

Software application 110 is configured, in at least one embodiment, to re-evaluate the priority assignments 144 any time there is a change in the number of currently existing undesired status conditions 142. That is, if an additional undesired condition is added to a currently existing undesired condition, or if the set of currently existing undesired conditions changes (in number or content), software application 110 re-evaluates the undesired conditions to determine which one (if there are multiple) of the undesired status conditions 142 has the highest priority assignment 144. After determining which undesired condition has the highest priority assignment, software application 110 instructs the display device 104 to then display (to the extent it wasn't previously displaying) the specific indicator 130 for the current undesired condition that has the highest priority assignment 144.

For example, if a patient support apparatus 20 currently has only the undesired condition of the litter frame 28 not being at its lowest height (low height condition), software application 110 will display a specific indicator 130 for this undesired condition, such as the particular indicator 130 shown in room NW23 of FIG. 4. If, at some point after this undesired height condition has occurred, the patient support apparatus 20 has its head section 40 moved lower than a desired angle such that the Fowler angle undesired condition occurs, software application 110 will compare the priority assignment 144 of the Fowler angle undesired condition to the priority assignment 144 of the low height undesired condition. It will then display the specific indicator 130 for whichever undesired condition has the higher priority. Using the examples of the priorities shown in FIG. 5, software application 110 will, in this example, stop displaying the specific indicator 130 for the low height condition, and will switch to displaying a specific indicator 130 for the Fowler angle condition (not shown in FIG. 4) and will add the generic indicator 132 with a numeric value of 1 for the low height undesired condition. Conversely, in the situation where both the low height undesired condition and the Fowler angle undesired condition are simultaneously occurring and display device 104 is displaying the specific indicator 130 for the Fowler angle, if the Fowler angle undesired condition ceases to exist (e.g. the Fowler section 40 is raised above a threshold angle), software application 110 will stop displaying the specific indicator 130 for the Fowler angle and will instead begin displaying the specific indicator 130 for the low height undesired condition. Software application 110 will also stop displaying the generic indicator 132 because only a single undesired condition remains currently active (the low height status condition 142).

In some embodiments, in addition to using the priority assignments 144 to determine what information to display on dashboard screen 120, software application 110 may be configured to also use the priority assignments 144 to determine what, if any, aural indications to make. For example, in some embodiments, software application 110 is configurable by an authorized user to only cause an aural alert to be issued by the display device 104 if an undesired condition occurs that has a priority value above a user-defined threshold. For example, if a user defines an aural alert threshold with a value of 5 or higher, then in the example shown in FIG. 5, display devices 104 will only issue an aural alert for the brake off undesired condition (priority value=5) and the Fowler angle undesired condition 142 (priority value=9). Any undesired status conditions 142 relating to the exit detection system 46 not being armed or not being armed with the right sensitivity level, the siderail(s) not being in a desired position, or the litter frame not being at a desired height, will not lead to the display device 104 issuing any aural alert.

In addition, software application 110 may be configured to not issue an additional aural alert for new undesired condition if the new undesired condition has a lower priority value than a currently existing undesired status condition 142 for which an aural alert has previously been issued. In the example show in FIG. 5, if software application 110 had caused a display device 104 to issue an aural alert for the Fowler angle of a patient support apparatus 20 not being within a desired range, and-while the Fowler angle was still out of its desired angular range-the brake on the patient support apparatus 20 was switched off, software application 110 would not instruct the display device 104 to issue another aural alert because the brake off status condition 142 (priority value=5) has a lower priority than the previously issued aural alert for the Fowler angle (priority value=9). However, if an aural alert was issued by a display device 104 for a patient support apparatus 20 not being in its lowest height, and subsequently the brake was switched off, software application 110 would instruct the display device 104 to issue another aural alert because the brake undesired condition has a higher priority value than the low height undesired condition.

In addition to, or in lieu of, using the priority assignment values 144 for determining what information is to be displayed on display devices 104 and/or what aural alerts are to be issued by display devices 104, software application 110 may be configured to use the priority assignment values 144 for determining what notifications to send and not to send to mobile devices carried by caregivers. The mobile devices may include any of the mobile display devices 104a, caregiver badges 118, and/or other devices carried by caregivers. As a result, software application 110 can be configured to send text alerts, emails, or other notifications to one or more mobile devices if an undesired condition occurs that has a priority assignment level higher than a user-specified threshold. If an undesired condition has a priority assignment level lower than the user-specified threshold, software application 110 can withhold the sending of such text alerts, emails, or other types of notifications.

By setting the priority assignments 144 to user-desired values, software application 110 helps avoid alarm fatigue for caregivers. This is done by reducing visual notifications on dashboard screen 120, by reducing aural alerts from display devices 104, and/or by reducing email, text, or other notifications sent to mobile devices when an undesired condition occurs that has a user-defined priority level that is less than a user-defined value. Lower priority alerts are therefore less visually intrusive (on dashboard 120), cause no aural alerting, and/or cause no remote notifications sent to mobile devices, thereby reducing the number of alerts or notifications that a caregiver is present with while performing his or her duties.

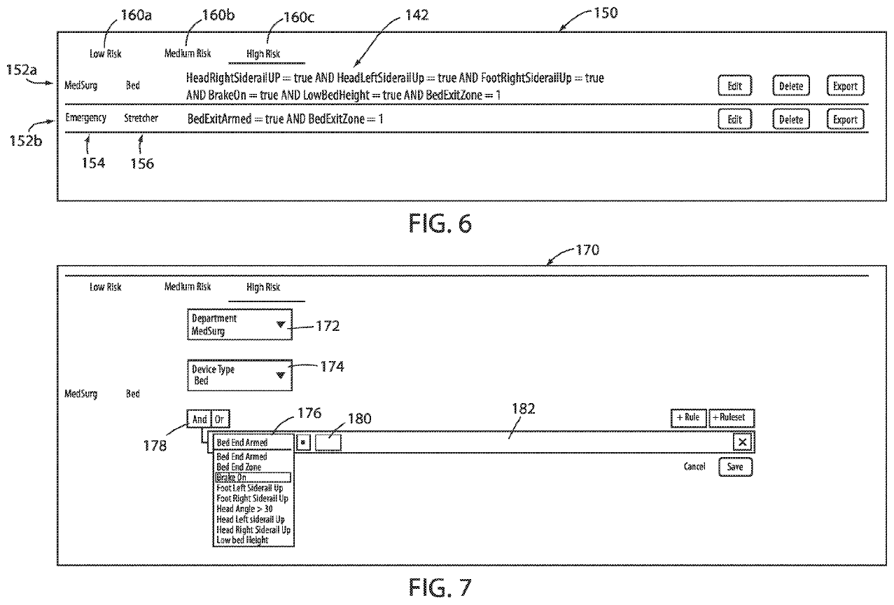

FIG. 6 illustrates one example of a rule summary screen 150 that may be displayed on a display of a user interface, such as computer 134, for authorized users of software application 110. Rule summary screen 150 lists the rules 152 that are currently defined by software application 110 for sending notifications to caregivers. In the example shown in FIG. 6, there are two rules defined: rule 152a and rule 152b. Each rule defines a set of desired bed status conditions 142 that must be satisfied in order to prevent software application 110 from notifying one or more caregivers, such as via showing an undesired condition on dashboard screen 120 (which, as discussed above, may be indicated on screen 120 using either a specific indicator 130 and/or a generic indicator 132), via sending an email or text, or via another method of notifying one or more caregivers. Thus, for example, the second rule 152b requires that exit detection system 46 is armed and that it is armed with the specific sensitivity level (exit zone) of 1. If either condition is untrue, software application 110 will notify one or more caregivers, such as by instructing one or more display devices 104 to show an undesired condition indicator 130 and/or 132 on dashboard screen 120 (and, if so configured, send a message to appropriate caregivers, such as via text, email, or other; and/or it may issue an aural alert).

Software application 110 is configured to allow authorized users to define rules 152 using any combination of Boolean logic of the status conditions 142 that are monitored by patient support apparatuses 20 and shared by the patient support apparatuses 20 with software application 110. Two examples of this Boolean logic are shown in the status condition column 142 of rules 152a and 152b. In addition to basing rules 152 on the status conditions 142 of patient support apparatuses 20, software application 110 is also configured to allow a user to base rules 152 on the location 154 of patient support apparatuses 20 and/or on the specific type 156 of individual patient support apparatuses 20 (such as whether the patient support apparatus 20 is a bed rather than a stretcher (or a cot), and/or the particular make or model of individual patient support apparatuses 20).

The location 154 of the patient support apparatus 20 may refer to the specific room in which the patient support apparatus 20 is located, a wing, department, floor, or other designated area within the healthcare facility. When a location 154 is added to a rule 152, software application 110 checks to see the location associated with a particular patient support apparatus 20 to see if a particular rule 152 applies to that patient support apparatus 20. Software application 110 checks for the locations of patient support apparatuses 20 using the locator IDs it receives from location beacons 84. That is, in addition to surveying the location of each location beacon 84 during their installation and inputting that information into software application 110 (or otherwise making it accessible to software application 110), software application 110 is configured to allow an authorized user to associate specific rooms (or other locations within the healthcare facility) with specific departments, wings, units, floors, and/or other locations. Therefore, for example, an authorized user can instruct software application that rooms NW1 through NW 10 (see FIG. 4.) belong to a first unit of the healthcare facility, rooms NW11 through NW20 belong to another unit, and rooms NW 21 through NW30 belong to still another unit. Alternatively, a user might instruct software application 110 that all of rooms NW1 through NW30 belong to a particular unit. Still other variations are, of course, possible.

The type 156 of a patient support apparatus 20, as noted, may refer to a make, model, or general type of patient support apparatus (e.g. stretcher versus bed). Software application 110 determines the types of patient support apparatuses 20 according to the unique ID 186 that it receives from each patient support apparatus 20. In such embodiments, the unique ID 186 includes information that identifies the particular make, model, and/or other type of each patient support apparatus 20.

Software application 110 is configured to include this make, model, and/or other type of information, or to have access to this information, so that it can discern what type of patient support apparatus 20 each of the patient support apparatuses 20 are that it receives status conditions from.

In addition to creating notification rules 152 based on the status conditions 142 (FIG. 6) of a patient support apparatus 20, the type 156 of patient support apparatus 20, and/or the location 154 of the patient support apparatus 20, software application 110 is configured to allow authorized users to create notification rules that are also based on one or more patient conditions, such as, but not limited to, the patient's fall risk 160. As shown in FIG. 6, the two notification rules 152a and 152b are both defined for patients who have been assessed to be high fall risks 160c. If a patient having a low or medium fall risk is present in a particular patient support apparatus 20, software application 110 will not use either of the notification rules 152a or 152b, but instead will use a rule 152 (not shown) that applies to the particular fall risk assessment for that particular patient.

Software application 110, in at least some embodiments, determines the fall risk of a particular patient by receiving this information from EMR server 98 and/or ADT server 94. ADT server 94 and/or EMR server 98 may also contain requirements data identifying one or more protocols that the healthcare facility requires its caregivers to follow when caring for one or more patients. Such requirements data, for example, may specify what assessments are to be performed on a patient, such as an assessment of the patient's fall risk and/or bed sore risk. Alternatively, such requirements data may be stored elsewhere, such as, but not limited to, the local rules of software application 110. In some embodiments, the requirements data that specifies which assessments (fall, skin, etc.) are to be performed for a given patient may depend upon the location of the patient within the healthcare facility. For example, some healthcare facilities may configure the local rules of software application 110 such that all patients within a particular wing, floor, or other section, receive both a fall risk assessment and a skin assessment, while patients within a different location are to receive only one or none of these assessments. Software application 110 automatically checks these local rules when a new patient is admitted to the healthcare facility (as determined from communication with ADT server 94) and, if no assessment is recorded in EMR server 98, it may be configured to display a reminder on dashboard screen 120 and/or send an alert to one or more mobile devices associated with the patient whose assessment has not been completed.

Thus, when a new patient enters the healthcare facility, software application 110 automatically determines from server 94 and/or its local rules if a particular patient is supposed to have a fall assessment, bed sore assessment, or other assessment performed. If so, software application 110 further sends an inquiry to EMR server 98 to determine if such an assessment has been completed for that particular patient. If it has not, software application 110 displays this lack of completion on dashboard screen 120. In the example shown in FIG. 4, the patients in room NW25 and NW 28 have not yet had a fall risk assessment performed, and this information is shown by the specific indicator 130 associated with a missing fall risk assessment. This particular specific indicator, in addition to showing a graphic of a patient falling, includes the textual information “no fall risk assessment.” Other types of graphics and/or text may, of course, be used.

Software application 110 receives the fall risk assessments of individual patients from EMR server 98 and/or from ADT server 94, and uses that information both when determining what rules 152 to apply to a particular patient's patient support apparatus 20, as well as when determining what information to display on dashboard screen 120. As was noted previously, software application 110 may be configured to display the background color of the header portions of 126 of each room icon 124 in a different color based on the corresponding patient's assessed fall risk.

FIG. 7 illustrates one example of a rule-setting screen 170 that may be displayed on a display device having appropriate access to software application 110, such as, but not limited to, one or more display devices 104 and/or computer 134 (FIG. 3). Rule-setting screen 170 allows a user to define the notification rules 152 that software application 110 is configured to follow. That is, rule-setting screen 170 allows an authorized user to create the rules 152, such as the rules 152a and 152b shown in FIG. 6. Rule-setting screen 170 includes a location selector 172, a type selector 174, a condition selector 176, a Boolean selector 178, a value selector 180, and a rule creation field 182. Location selector 172 allows the user to define whether a particular rule 152 will be based on a patient support apparatus's location within the healthcare facility or not. As was mentioned previously, the location may refer to not only specific rooms within the healthcare facility, but may also refer to particular departments, wings, units, or other organizational groupings of rooms within the healthcare facility. Location selector 172 allows the user to select whatever one of these organizational groupings that is to be applicable for a given rule, as well as to select a general location that encompasses the entire healthcare facility. In other words, if the user does not want a particular rule 152 to be limited to a particular location within the healthcare facility, location selector 172 includes an “entire healthcare facility” option, or the like, that allows the user to create a rule 152 that is not location specific.

Type selector 174 (FIG. 7) allows the user to create a rule that is specific to one or more particular types of patient support apparatuses 20 (or that is generic to all types). As was noted, the particular types may refer to beds, stretchers, cots, or other general types of patient support apparatuses 20. Alternatively, or additionally7, the particular types may refer to specific makes and/or models of beds, stretchers, etc. The type selector may also, or additionally, refer to patient support apparatuses 20 that have specific features and/or functions, such as, for example, patient support apparatuses 20 that include mattress therapy functions, or other functions that may not be present on all of the patient support apparatuses 20 within a given healthcare facility.

Condition selector 176 allows the user to select one or more status conditions 142 that are to be monitored as part of a rule 152. Condition selector 176 therefore includes all of the status conditions 142 that patient support apparatuses 20 are capable of monitoring and reporting to software application 110 (via network transceiver 60). These include, but are not limited to, the armed/disarmed status of exit detection system 46, the specific zone (i.e. sensitivity level) at which the exit detection system 46 is currently armed, the activated/deactivated status of the brake, the position of each siderail 36, the angular orientation of Fowler section 40, the height of litter frame 28, the state of an onboard monitoring system (armed or disarmed), the state of an AC power cable (plugged in or unplugged), the state of nurse call cable 78, the state of the communication channel (established/not established) between patient support apparatus 20 and the healthcare facility's nurse call system, the state of a mattress 38 onboard patient support apparatus 20 (including any therapies being implemented using the mattress 38), the weight of any objects on patient support apparatus 20, the presence/absence of the patient onboard patient support apparatus 20 (as detected by scale system 46, or by other means), the state of any batteries onboard patient support apparatus 20 or location beacon 84, the servicing state of any electrical and/or mechanical components of patient support apparatus 20, and/or other status conditions relating to patient support apparatus 20.

Boolean selector 178 (FIG. 7) allows the user to select a particular Boolean relationship that must be satisfied as part of the rule 152. Boolean selector 178 therefore includes options such as TRUE, FALSE, although it will be understood that additional Boolean operators may be included as part of selector 178. Value selector 180 allows the user to select a particular value that must be satisfied as part of a rule 152. Value selector 180 includes the values for the different sensitivity levels of exit detection system 46 and, in some embodiments, values for different desired angular thresholds for Fowler section 40. Still other values may also or alternatively be included within value selector 180.

Rule creation field 182 (FIG. 7) is where the user creates a particular rule 152 using the previously described selectors. Two examples of the types of rules 152 that may be created in rule creation field 182 are rules 152a and 152b shown in FIG. 6. Other specific rules 152 may also be created. After a particular rule is created, the user may save the rule within software application 110. Once saved, the rule can be applied to patient support apparatuses 20, edited, and/or stored for potential future usage. As was noted, once a rule is saved and applied, software application 110 is configured to monitor the incoming status data received from the patient support apparatuses 20 within the healthcare facility and check to see if the conditions of any applicable rules 152 are met. If so, software application 110 is configured to provide notification to the appropriate caregivers, such as by changing the data displayed on dashboard screen 120 and/or by providing aural or other notifications to one or more caregivers.

In addition to, or in lieu of, displaying the specific indicators 130 and/or the generic indicators 132 on dashboard screen 120, software application 110 may be configured to display information regarding the recordation of a patient's weight. That is, in some embodiments, software application 110 is also configured to display on dashboard 120 information indicating whether or not a patient's weight has been recorded, and in some cases, whether or not a patient's weight has been recorded within a time frame desired by that particular healthcare facility. When a patient's weight is recorded using scale system 46 of patient support apparatus 20, the fact of this weight recordation may be communicated to software application 110 in one (or both) of two manners. In a first manner, whenever a caregiver uses the onboard scale system 46 to take a patient weight reading, controller 48 of patient support apparatus 20 sends a message to software application 110 indicating that the patient's weight was recorded. The message may also include the date and time of the weight recording. Alternatively, software application 110 may record the time and date at which the message is received.

Alternatively, or additionally, software application 110 may be configured to retrieve the patient's weight recordations from EMR server 98. The readings of the patient's weight in EMR server 98 include a time and date for each weight recording (if any are recorded). As was discussed previously, software application 110 may be configured to automatically determine which particular patient is assigned to a particular patient support apparatus 20 by using the association information of patient support apparatuses 20 to rooms (via location beacons 84) and the association information of patients to rooms (via ADT server 94), or by other means. Once software application 110 knows which patient is assigned to which patient support apparatus 20, it can determine from EMR server 98 whether that particular patient's weight has been recorded (and when). Software application 110 can therefore receive patient weight reading information (time of reading, date of reading, weight reading, etc.) from patient support apparatuses 20 directly, from EMR server 98, or from both sources.

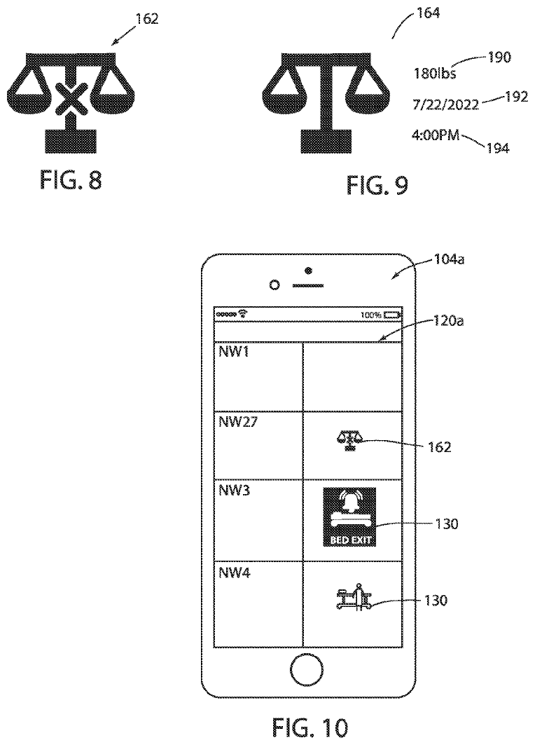

In those embodiments where software application 110 is configured to receive patient weight readings, software application 110 may be configured to display information about these weight readings on dashboard screen 120. More specifically, in such embodiments, software application 110 may be configured to instruct display device 104 to display a missing-weight icon 162 (FIG. 8) and/or a weight-recorded icon 164 (FIG. 9). Software application 110 may be configured to display missing-weight icon 162 whenever a patient's weight is not recorded within either of two potentially different time periods: a first time period measured from the time the patient is assigned to a room (or otherwise admitted to the healthcare facility), and a second time period measured from the patient's last recorded weight reading. In other words, many healthcare facilities have internal protocols requiring that a patient's weight be recorded within a desired time period after they are initially admitted to the hospital. Many healthcare facilities also require that the patient's weight be re-recorded periodically thereafter at desired intervals. In some healthcare facilities, these time periods are the same, while in other healthcare facilities, these two time periods may be different. Software application 110 is configured to allow the user to define these two time periods differently or the same, thereby allowing users to define how soon a patient's weight is to be recorded upon initial admittance to the healthcare facility and how frequently the patient's weight is to be updated (i.e. re-taken) after the initial weight reading is recorded.

Software application 110 is configured to instruct the display device 104 to display the missing-weight icon 162 if the patient's weight hasn't been taken within either of these two time periods. That is, if a recently admitted patient has not had his or her weight recorded within the first time period, software application 110 is configured to instruct the display device 104 to display the missing-weight icon 162 on dashboard screen 120. Similarly, if a patient whose weight was previously recorded doesn't have their weight re-measured within the second time period of their last weight reading, software application 110 is also configured to instruct the display device 104 to display the missing-weight icon 162. Software application 110 is therefore configured to display the missing-weight icon 162 whenever the patient's weight hasn't been recorded within the desired first or second time period. Missing-weight icon 162 therefore reminds the caregiver that a weight reading for a particular patient is overdue.

Software application 110 is configured to instruct the display device 104 to display the weight-recorded icon 164 (FIG. 9) whenever a patient's weight has been recorded within the first or second time periods discussed above. That is, software application 110 is configured to instruct the display device 104 to display the weight-recorded icon 164 whenever a time weight reading for a patient is on record. Caregivers who see the weight-recorded icon 164 therefore know that the healthcare facility's protocols for patient weight readings is currently in compliance for that particular patient.

In some embodiments, software application 110 is configured to not only display the weight-recorded icon 164 in each room icon 124 for which a patient's weight reading has been timely recorded, but also to display a weight reading value 190, a date of the weight reading 192, and/or a time of the weight reading 194. The display of this information not only confirms that a weight reading has been taken in a timely manner for this patient, but also lets the caregiver know the value of the weight reading and when it was taken.

As shown in FIG. 4, software application 110 may be configured to instruct display devices 104 to enlarge the size of room icons 124 for any rooms in which an exit detection event has been detected by exit detection system 46. Thus, in the example shown in FIG. 4, room NW3 and room NW18 are displayed in a larger size because a patient exit has been detected by exit detection system 46. Software application 110 may be configured to allow the user to customize the background color of room icon 124 for situations when such a patient exit event is detected. For example, display device 104 may display the enlarged room icons for a patient exit event with a red background. The user, of course, can choose other colors for the background (and in some cases, the foreground color of the exit detection specific indicator 130) of room icons 124 when a patient exit is detected. Additionally, in some embodiments, software application 110 may be configured to allow the user to customize how large a room icon 124 is enlarged in respond to patient exit detections, whether a room icon 124 is enlarged in response to other undesired conditions, and/or in other manners.

In some embodiments, software application 110 displays the background color of a room icon 124 in which exit detection system 46 is not armed (but is desirably armed, according to one or more rules 152) with a different color than the background colors of the other room icons 124. In the example shown in FIG. 4, display device 104 is instructed by software application 110 to display the background colors of the room icons for rooms NW14 and NW17 with a red background because the exit detection systems 46 of the patient support apparatuses 20 in these rooms is not armed. This red background color may be customized by the user of software application 110 to be a different color.

The specific indicator 130 of rooms NW14 and NW17 (FIG. 4) indicates that the exit detection systems 46 of these two patient support apparatuses 20 are not armed, and that the rules 152 in place for these two patient support apparatuses 20 require the exit detection system 46 to be armed. In some embodiments, software application 110 is configured such that a user can customize the color of these specific indicators 130 (and/or any of the other specific indicators 130).

In addition to providing specific indicators 130 for exit detection system 46 not being armed, software application 110 may also provide a specific indicator 130 when a rule 152 dictates that a specific sensitivity level of exit detection system 46 be used and that particular sensitivity level is not currently selected. An example of this specific indicator 130 is shown for room NW16 in FIG. 4. Many exit detection systems are configured to allow a user to select different zones of permitted movement. The different zones allow a patient to move different amounts before the exit detection system issues an alert. In the example of FIG. 4, the patient support apparatus 20 includes an exit detection system 46 having multiple zones and, although the exit detection system 46 is armed, it is currently armed with the wrong zone (as dictated by the applicable rule 152). Further information about the zones and/or operation of an exit detection system that may be incorporated into patient support apparatus 20 and utilized in caregiver assistance system 106 are found in commonly assigned U.S. patent application Ser. No. 14/918,003 filed Oct. 20, 2015, by inventors Marko Kostic et al. and entitled EXIT DETECTION SYSTEM WITH COMPENSATION, the complete disclosure of which is incorporated herein by reference.

Although a typical mobile display device 104a may be associated with a particular caregiver, this is generally not true for stationary display devices 104b. Stationary display devices 104b, which may include large screen smart televisions, may be associated with a particular unit of a healthcare facility, a particular nurse's station, wing, floor, and/or other section of the healthcare facility. For these devices, the login credentials may be tailored to the particular location and/or intended function of that particular stationary display device 104b. For example, a stationary display device 104b may be associated with an oncology unit, an east wing, nurse's station XYZ, the second floor, or rooms A through G, or something else. In such instances, software application 110 may be configured to assign a username and password to each such display device 104b that is custom tailored to that specific device. Thus, for example, if a particular display device 104b is positioned at a nurse's station within a pediatric oncology unit, the display device 104b may be assigned a username of “pediatric oncology display” and have its own specific password. Once an authorized user has logged into software application 110 via that device, caregiver assistance application displays the rooms and/or patient data corresponding to the pediatric oncology unit on that particular device 104b. The room and/or patient data may include rooms and/or patients that are assigned to multiple caregivers, thereby allowing the display device 104b to display information beyond that associated with a single caregiver.

Dashboard screen 120 (FIG. 4) is typically—though not necessarily always—displayed on the display of stationary electronic devices 104b, rather than mobile displays 104a. This is because stationary display devices 104b typically have larger sized displays that mobile display devices 104a, and dashboard screen 120 includes a large amount of information that may be difficult to read on a mobile display device 104a having a relatively small screen. Accordingly, software application 110 may be configured to send display instructions to mobile display devices 104a that are different than the display instructions that it sends to stationary display device 104b. More particularly, software application 110 may be configured to instruct mobile display devices 104a to an abbreviated dashboard screen that contains room information for only the rooms to which a particular caregiver is assigned responsibility. One example of an abbreviated dashboard screen 120a is shown in FIG. 10.

Abbreviated dashboard screen 120a displays the status of patient support apparatuses 20 for a more limited number of rooms than dashboard screen 120. In the example of FIG. 10, abbreviated dashboard screen 120a display the status for only four rooms (NW1, NW27, NW3, and NW4). Abbreviated dashboard screen 120a may display different rooms, as well as different numbers of rooms. In general, software application 110 instructs mobile display devices 104a to display only those rooms on dashboard screen 120a to which a caregiver has been assigned. Accordingly, in the example of FIG. 10, the particular mobile display device 104a shown therein is associated with a caregiver who is responsible for the patients in the four specific rooms displayed on abbreviated dashboard screen 120a (i.e. rooms NW1, NW27, NW3, and NW4). A different caregiver in the healthcare facility who is assigned to patients in different rooms would have a different abbreviated dashboard screen 120a displayed on their mobile display device 104a.

Abbreviated dashboard screen 120a includes a specific indicator 130 for those rooms in which an undesired condition for the patient support apparatus 20 currently exists. Although not shown in the example of FIG. 10, software application 110 may be configured to also display a generic indicator 132 on abbreviated dashboard screen 120a if a patient support apparatus 20 within a given room has more than one undesired condition. In other words, software application 110 may be configured to instruct mobile display devices 104a to display specific indicators 130 and generic indicators 132 in the same manner that it instructs stationary display devices 104b to display specific indicators 130 and generic indicators 132, as was discussed above.

Software application 110 may be configured to automatically determine which rooms a particular caregiver has been assigned by communicating with a server on local area network 74 that maintains room assignments for caregivers. In the example illustrated in FIG. 3, nurse call server 96 is shown to include a caregiver-room assignment table 122 that stores the room assignments for the caregivers within the healthcare facility. Caregiver-room assignment table 122 may also, or alternatively, be stored on a different server. During installation of software application 110, an authorized administrator inputs the IP address of the server containing caregiver room assignment table 122 (and/or other data necessary to gain access to caregiver-room assignment table 122). Similar data is also input for all of the other servers and tables discussed herein. After a particular user successfully logs into software application 110, software application 110 sends a message to the server having caregiver room assignment table 122. The message requests an up-to-date listing of the rooms that are assigned to the specific caregiver who has just logged in. After receiving this information, software application 110 may instruct the mobile display device 104a to only display those rooms on the display of the mobile display device 104a that have patients assigned to that particular caregiver.

The data displayed in dashboard screens 120 and 120a is updated in real time, or near real time. In most embodiments of patient support apparatuses 20, the patient support apparatuses 20 are configured to automatically (and nearly immediately) communicate their status to patient support apparatus server 86 whenever a change occurs in their status. Thus, for example, if the nurse call cable 78 gets unplugged from the nurse call outlet 82, the patient support apparatus 20 sends a message automatically and almost immediately thereafter to patient support apparatus server 86. Software application 110 of patient support apparatus server 86 automatically, and immediately or nearly immediately, forwards this status update to all of the display devices 104 are currently displaying status information for that particular room (or that are able to navigate to a page, such as on a mobile display device 104a, that displays that information). A caregiver, who may be remote from a particular room and/or a particular patient support apparatus 20, but nearby to a display device 104, thereby gets a real time, or near real time, update of the status of patient support apparatus 20 when utilizing software application 110.

In addition to communicating with display devices 104, software application 110 may be configured to also communicate with caregiver badges 118 (FIG. 3). Software application 110 may communicate any of the information shown in dashboard screens 120 and/or 120a to one or more caregiver badges 118. Software application 110 may also communicate aural alerts and/or other types of notifications to badges 118. That is, as was mentioned, in some embodiments, software application 110 may be configured to only communicate undesired conditions to badges 118 if the undesired conditions have a priority assignment value 144 that is above a user-defined threshold. Thus, in some embodiments, a user can configure software application 110 to only send notifications to badges 118 for undesired conditions that meet a desired level or priority, and to not send such notifications to badges 118 for undesired conditions that have a priority level less than the desired priority level.

In such embodiments, it may be easier to provide alerts to the caregiver by having the electronic device vibrate, emit an audible sound, and/or illuminate one or more lights on the device. Such alerts may be more difficult to communicate to a caregiver when caregiver assistance system 106 is implemented using browser-connected display devices 104, particularly if the caregiver has the browser application closed and/or running in the background and/or is not looking at the information currently being displayed on the screen of the display device 104. Such native applications may be programmed for execution with the Android or iOS operating systems, or still other operating systems utilized by the display device 104.

It will also be understood that software application 110 may be configured to instruct display devices 104 to display the above-described information in different manners. For example, in some embodiments, software application 110 sends the data defining the graphics shown in dashboard screens 120 and 120a to the corresponding display device 104 and instructs the display device 104 to display those graphics. However, in other examples, some or all of the graphics shown in the dashboard screens 120 and/or 120a may be stored locally in a software application executed by the display device 104 and software application 110 may instruct the display device 104 to display these graphics without having to forward these graphics to the display device. Still other manners of instructing the display devices 104 what to display may also, or additionally, be used.

Various additional alterations and changes beyond those already mentioned herein can be made to the above-described embodiments. This disclosure is presented for illustrative purposes and should not be interpreted as an exhaustive description of all embodiments or to limit the scope of the claims to the specific elements illustrated or described in connection with these embodiments. For example, and without limitation, any individual element(s) of the described embodiments may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Any reference to claim elements in the singular, for example, using the articles “a,” “an,” “the” or “said,” is not to be construed as limiting the element to the singular.

1. A software application embodied in a non-transitory computer readable medium and adapted, when executed by a processor of a computing device, to cause the computing device to perform the following:
receive status conditions from a patient support apparatus, each of the status conditions including a current state of a component of the patient support apparatus;
use the status conditions to determine a set of undesired conditions that currently exist for the patient support apparatus;
receive priority assignments for the set of undesired conditions from a user interface;
if the set of undesired conditions contains multiple undesired conditions that currently exist for the patient support apparatus, perform the following:
(a) select the undesired condition having the highest priority assignment from the set of undesired conditions;
(b) instruct a display device to display a specific indicator that specifically identifies the selected undesired condition; and
(c) instruct the display device to display a generic indicator that does not specifically identify the undesired conditions in the set that do not have the highest priority assignment; and
if the set of undesired conditions contains only a single undesired condition that currently exists for the patient support apparatus, instruct the display device to display a single specific indicator that specifically identifies the single undesired condition.

2. The software application of claim 1 wherein the generic indicator is a number having a value equal to one less than a total number of undesired conditions in the set of undesired conditions; the specific indicator includes a graphical symbol; the set of undesired conditions includes at least one of the following: a brake on the patient support apparatus not set, a position of one or more siderails on the patient support apparatus not being in a desired position, an exit detection system of the patient support apparatus not being armed, or a height of a litter frame of the patient support apparatus not being at a desired height; and the software application is further adapted to instruct the computer device to receive location data from the patient support apparatus, to use the location data to determine a room number in which the patient support apparatus is currently located, and to instruct the display device to display the room number on the display device.

3-5. (canceled)

6. The software application of claim 1 wherein the software application is further configured to instruct the computer device to perform the following:
receive additional status conditions from a plurality of additional patient support apparatuses, the additional status conditions including a current state of a plurality of components of each of the plurality of additional patient support apparatuses;
use the additional status conditions to determine an additional set of undesired conditions that currently exist for each one of the plurality of additional patient support apparatuses; and
for each additional patient support apparatus in the plurality of additional patient support apparatuses, if the additional set contains multiple undesired conditions that currently exist for the additional patient support apparatus,
(a) select the undesired condition having the highest priority assignment from the additional set of undesired conditions;
(b) instruct the display device to display the specific indicator that specifically identifies the selected undesired condition; and
(c) instruct the display device to display the generic indicator that does not specifically identify the undesired conditions in the additional set that do not have the highest priority assignment; and
if the additional set of undesired conditions contains only a single undesired condition that currently exists for the additional patient support apparatus, instruct the display device to display a single specific indicator that specifically identifies the single undesired condition.

7. The software application of claim 1 wherein the computing device is a server communicatively coupled to the display device by a computer network; the server is communicatively coupled to the display device by a WiFi connection; the display device is one of a smart phone, a tablet computer, a television, or a laptop computer; and wherein the software application is further adapted to instruct the display device to display an enclosed area on a display of the display device and to display both the generic indicator and the specific indicator within the enclosed area, wherein the enclosed area corresponds to a particular room of a healthcare facility.

8-11. (canceled)

12. The software application of claim 1 wherein the software application is further adapted to instruct the computing device to send an alert to a caregiver badge, wherein the alert notifies a caregiver associated with the caregiver badge of the selected undesired condition but does not notify the caregiver of the undesired conditions in the set that do not have the highest priority assignment.

13. (canceled)

14. The software application of claim 1 wherein the software application is further adapted to instruct the computing device to receive undesired condition definitions that are based on at least one of the following: a current location of the patient support apparatus, a department to which the patient support apparatus is assigned, a health condition of a patient, or a patient support apparatus type.

15-17. (canceled)

18. The software application of claim 1 wherein the software application is further adapted to instruct the computing device to perform the following: (1) receive weight data indicating when a weight of a patient assigned to the patient support apparatus was last measured using the patient support apparatus, and (2) instruct the display device to display a weight icon based on the weight data.

19-22. (canceled)

23. A software application embodied in a non-transitory computer readable medium and adapted, when executed by a processor of a computing device, to cause the computing device to perform the following:
receive status conditions from a patient support apparatus, the status conditions including a current state of a plurality of components of the patient support apparatus;
receive weight data indicating when a weight of a patient assigned to the patient support apparatus was last measured using the patient support apparatus; and
instruct a display device to display a specific indicator based on the status conditions and a weight icon based on the weight data.

24. The software application of claim 23 wherein the software application is adapted to select the weight icon from a missing-weight icon and a weight-recorded icon, and the software application is adapted to instruct the computing device to perform the following:
receive patient assignment data indicating when a new patient is assigned to the patient support apparatus;
if the computing device does not receive the weight data within a time period after receiving the patient assignment data, instruct the display device to display the missing-weight icon; and
if the computing device does receive the weight data with the time period after receiving the patient assignment data, instruct the display device to display the weight-recorded icon.

25. The software application of claim 24 wherein the software application is further adapted, after receiving the weight data, to instruct the computing device to perform the following:
determine if updated weight data has been received within a time window of receiving the weight data;
if the computing device has not received the updated weight data within the time window, instruct the display device to display the missing-weight icon; and
if the computing device has received the updated weight data with the time window, instruct the display device to display the weight-recorded icon.

26-30. (canceled)

31. The software application of claim 23 wherein the software application is further adapted to instruct the computing device to receive the weight data from at least one of an electronic medical records server or the patient support apparatus.

32. The software application of claim 23 wherein the software application is further adapted to instruct the computing device to perform the following:
use the status conditions to determine a set of undesired conditions that currently exist for the patient support apparatus;
receive priority assignments for the set of undesired conditions from a user interface;
if the set contains multiple undesired conditions that currently exist for the patient support apparatus, perform the following:
(a) select the undesired condition having the highest priority assignment from the set of undesired conditions;
(b) instruct the display device to display the specific indicator that specifically identifies the selected undesired condition; and
(c) instruct the display device to display a generic indicator that does not specifically identify the undesired conditions in the set that do not have the highest priority assignment; and
if the set of undesired conditions contains only a single undesired condition that currently exists for the patient support apparatus, instruct the display device to display a single specific indicator that specifically identifies the single undesired condition.

33-35. (canceled)

36. The software application of claim 32 wherein the software application is further adapted to instruct the computer device to receive location data from the patient support apparatus, to use the location data to determine a room number in which the patient support apparatus is currently located, and to instruct the display device to display the room number on the display device.

37. The software application of claim 32 wherein the software application is further configured to instruct the computer device to perform the following:
receive additional status conditions from a plurality of additional patient support apparatuses, the additional status conditions including a current state of a plurality of components of each of the plurality of additional patient support apparatuses;
use the additional status conditions to determine an additional set of undesired conditions that currently exist for each one of the plurality of additional patient support apparatuses; and
for each additional patient support apparatus in the plurality of additional patient support apparatuses, if the additional set contains multiple undesired conditions that currently exist for the additional patient support apparatus,
(a) select the undesired condition having the highest priority assignment from the additional set of undesired conditions;
(b) instruct the display device to display the specific indicator that specifically identifies the selected undesired condition; and
(c) instruct the display device to display the generic indicator that does not specifically identify the undesired conditions in the additional set that do not have the highest priority assignment; and
if the additional set of undesired conditions contains only a single undesired condition that currently exists for the additional patient support apparatus, instruct the display device to display a single specific indicator that specifically identifies the single undesired condition.

38-49. (canceled)

50. A software application embodied in a non-transitory computer readable medium and adapted, when executed by a processor of a computing device, to cause the computing device to perform the following:
receive status conditions from a patient support apparatus, the status conditions including a current state of a plurality of components of the patient support apparatus;
receive undesired condition definitions from a user interface that are based on at least one of the following: a current location of the patient support apparatus, a department to which the patient support apparatus is assigned, or a patient support apparatus type;
use the undesired condition definitions and the status conditions to determine if an undesired condition currently exists for the patient support apparatus;
if an undesired condition currently exists for the patient support apparatus, instruct a display device to display information indicative of the undesired condition; and
if an undesired condition does not currently exist for the patient support apparatus, not instruct the display device to display information indicative of the undesired condition.

51. The software application of claim 50 wherein the software application is further adapted to instruct the computing device to receive undesired condition definitions that are based on both the department to which the patient support apparatus is assigned and the patient support apparatus type.

52. The software application of claim 50 wherein the patient support apparatus type defines whether the patient support apparatus is a bed or a stretcher.

53. The software application of claim 50 wherein the patient support apparatus type defines a specific model of a bed or a specific model of a stretcher.

54. The software application of claim 50 wherein the software application is further adapted to instruct the computing device to perform the following:
receive priority assignments for a plurality of undesired conditions from the user interface;
use the status conditions to determine a set of undesired conditions that currently exist for the patient support apparatus;
if the set contains multiple undesired conditions that currently exist for the patient support apparatus, perform the following:
(a) select the undesired condition having the highest priority assignment from the set of undesired conditions;
(b) instruct the display device to display the information indicative of the undesired condition by displaying a specific indicator that specifically identifies the selected undesired condition; and
(c) instruct the display device to display the information indicative of the undesired condition by displaying a generic indicator, wherein the generic indicator does not specifically identify the undesired conditions in the set that do not have the highest priority assignment; and
if the set of undesired conditions contains only a single undesired condition that currently exists for the patient support apparatus, instruct the display device to display the information indicative of the undesired condition by displaying a single specific indicator that specifically identifies the single undesired condition.

55-65. (canceled)

66. The software application of claim 50 wherein the software application is further adapted to instruct the computing device to perform the following: (1) receive patient assignment data indicating when a new patient is assigned to the patient support apparatus; (2) receive weight data indicating when a weight of a patient assigned to the patient support apparatus was last measured using the patient support apparatus; (3) if the computing device does not receive the weight data within a time period after receiving the patient assignment data, instruct the display device to display a missing-weight icon; and (4) if the computing device does receive the weight data with the time period after receiving the patient assignment data, instruct the display device to display a weight-recorded icon.

67-75. (canceled)
